(19) United States
(12) Patent Application Publication
(10) Pub. No.: US 2023/0406824 A1
(43) Pub. Date: Dec. 21, 2023

(54) TRYPTAMINE DERIVATIVES AND THEIR THERAPEUTIC USES

(71) Applicant: CAAMTECH, INC., lssaquah, WA (US)

(72) Inventor: Andrew R. CHADEAYNE, lssaquah, WA (US)

(73) Assignee: CAAMTECH, INC., lssaquah, WA (US)

(21) Appl. No.: 18/248,577

(22) PCT Filed: Oct. 12, 2021
(86) PCT No.: PCT/US2021/054534

§ 371 Date: Apr. 11, 2023

Related U.S. Application Data
(60) Provisional application No. 63/226,954, filed on Jul. 29, 2021.
(60) Provisional application No. 63/135,144, filed on Jan. 8, 2021.
(60) Provisional application No. 63/090,930, filed on Oct. 13, 2020.

(51) Int. Cl. C07D 209/16; A61K 45/06

(52) U.S. Cl. CPC C07D 209/16; A61K 45/06

(57) ABSTRACT

This disclosure relates to tryptarmes derivatives, compositions and pharmaceutical compositions containing them as well as their use in treating various diseases.

### CROSS-REFERENCE TO RELATED APPLICATIONS

This application claims priority to U.S. Provisional Application No. 63/090,930, filed on Oct. 13, 2020, to U.S. Provisional Application No. 63/135,144, filed on Jan. 8, 2021, and to U.S. Provisional Application No. 63/226,954, filed on Jul. 29, 2021, the disclosures of which are incorporated by reference.

### TECHNICAL FIELD

This disclosure relates to tryptamines derivatives, compositions and pharmaceutical compositions containing them as well as their use in treating various diseases.

### BACKGROUND OF THE INVENTION

N,N-dimethyltryptamine (DMT) and its derivatives have been used by humans for centuries because of their psychoactive, entheogenic, or hallucinogenic effects, or combinations thereof (Cameron & Olson, 2018). Psilocybin, the 4-phosphate variant of DMT, is arguably its most studied derivative. Psilocybin is one of several naturally occurring psychoactive tryptamines found in “magic” mushrooms. When consumed by humans, psilocybin serves as a prodrug of psilocin. Upon digestion, psilocybin hydrolyses to generate psilocin, the 4-hydroxy derivative of DMT. Psilocin is a potent serotonin 2a-agonist, which is responsible for its psychoactive properties (Dinis-Oliveira, 2017; Nichols, 2012).

Psychoactive tryptamines like DMT and psilocin have garnered significant interest recently because of their potential for treating mood disorders, including depression, anxiety, addiction, and post-traumatic stress disorder (PTSD) (Johnson & Griffiths, 2017; Carhart-Harris & Goodwin, 2017). Altering the chemical structure within this class of compounds can dramatically influence the potency and action of the drugs. For example, merely changing the N,N-dialkyl groups on DMT can modify its psychoactive properties: increasing the chain length of the two alkyl groups of the tryptamine to larger than n-butyl dramatically reduces or eliminates the psychoactive effects (Bradley & Johnston, 1970).

New psychoactive tryptamines have been identified in “magic mushrooms” as recently as 2017. (Lentz, et al., 2017.) Until this year, there was no general synthetic method for producing useful amounts of the minor psychoactive tryptamines. (Sherwood, Halberstadt, et al.) One of these minor components is aeruginascin (Jensen, et al., 2006), the N-trimethyl analogue of psilocybin. The limited exposure of humans to Inocybe aeruginascens mushrooms, the only known species in which aeruginascin has been found, has resulted in hallucinations that exhibited only euphoric experiences. (Gartz, 1989). This is in contrast to psilocybin and psilocin mushrooms, which often lead to dysphoric moods during the psychedelic experience. Despite these observations, the pharmacological activity of aeruginascin has remained unexplored.

Even with this previous work, there is a need to develop new psilocybin derivatives with improved properties for treatment of psychological disorders.

### SUMMARY OF THE INVENTION

The invention relates to a compound of formula (I):

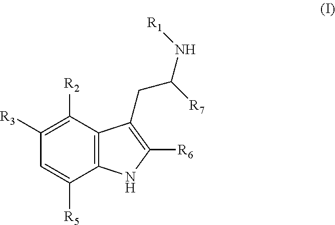

wherein
R₁ is a straight chain or branched C₁-C₆ alkyl or a straight chain or branched C₂-C₆ alkenyl;
R₂ and R₃ are independently chosen from hydrogen, hydroxyl, —OR₉, —OC(O)R₈, or —OC(O)OR₄, —OSO₂R₄;
R₄ is a straight chain or branched C₁-C₆ alkyl or a substituted or unsubstituted aryl; and
R₅, R₆ and R₇ are each independently hydrogen or a straight chain or branched C₁-C₆ alkyl; or a pharmaceutically acceptable acid-addition salt thereof.

This invention also relates to a compound of formula (Ia):

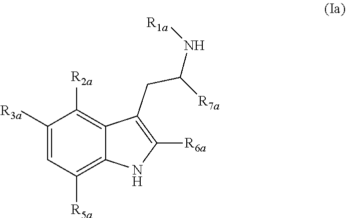

R₁ₐ is a straight chain or branched C₁-C₆ alkyl or a straight chain or branched C₂-C₆ alkenyl;
one of R₂ₐ and R₃ₐ is hydrogen and the other of R₂ and R₃ is selected from —OC(O)R₈ₐ, —OC(O)OR₄ₐ, and —OSO₂R₄ₐ;
R₄ₐ is a straight chain or branched C₁-C₆ alkyl or a substituted or unsubstituted aryl;
R₈ₐ is selected from straight chain or branched C₁-C₆ alkyl;
R₅ₐ, R₆ₐ and R₇ₐ are each independently hydrogen or a straight chain or branched C₁-C₆ alkyl; or a pharmaceutically acceptable acid-addition salt thereof.

This invention also relates to a compound of formula (II):

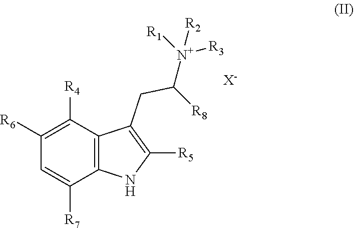

wherein
R₁ is a straight chain or branched C₁-C₆ alkyl or a straight chain or branched C₂-C₆ alkenyl;
R₂ and R₃ are both hydrogen;
R₄ and R₆ are independently chosen from hydrogen, hydroxyl, —OR₅, —OC(O)R₁₁, —OC(O)OR₁₂, —OSO₂R₁₂;
R₅ is a straight chain or branched C₁-C₆ alkyl or a substituted or unsubstituted aryl;
R₁₁ is a straight chain or branched C₁-C₆ alkyl or a substituted or unsubstituted aryl;
R₁₂ is a straight chain or branched C₁-C₆ alkyl or a substituted or unsubstituted aryl;
R₇, R₈ and R₉ are each independently hydrogen or a straight chain or branched C₁-C₆ alkyl; and
X⁻ is a pharmaceutically-acceptable anion.

This invention also relates to a compound of formula (IIa):

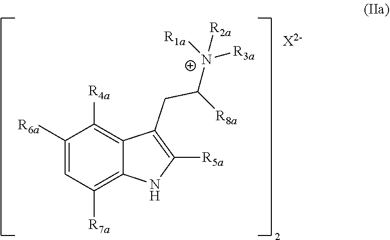

wherein
R₁ₐ is a straight chain or branched C₁-C₆ alkyl or a straight chain or branched C₂-C₆ alkenyl;
R₂ₐ and R₃ₐ are both hydrogen;
R₄ₐ and R₆ₐ are independently chosen from hydrogen, hydroxyl, —OR₅, —OC(O)R₁₁ₐ, —OC(O)OR₁₂ₐ, —OSO₂R₁₂ₐ;
R₅ₐ is a straight chain or branched C₁-C₆ alkyl or a substituted or unsubstituted aryl;
R₁₁ₐ is a straight chain or branched C₁-C₆ alkyl or a substituted or unsubstituted aryl;
R₁₂ₐ is a straight chain or branched C₁-C₆ alkyl or a substituted or unsubstituted aryl;
R₇ₐ, R₈ₐ and R₉ₐ are each independently hydrogen or a straight chain or branched C₁-C₆ alkyl; and
X²⁻ is a pharmaceutically-acceptable dianion.

The invention relates to compositions comprising, consisting essentially of, or consisting of a compound of formula (I), formula (Ia), formula (II), or formula (IIa), and an excipient. The invention also relates pharmaceutical compositions comprising a therapeutically effective amount of a compound of formula (I), formula (Ia), formula (II), or formula (IIa), wherein the excipient is a pharmaceutically acceptable carrier. The invention further relates to a method of preventing or treating a psychological disorder comprising the step of administering to a subject in need thereof a therapeutically effective amount of a compound of formula (I), formula (Ia), formula (II), or formula (IIa) or of a pharmaceutical composition containing the compound.

The invention also relates to a composition comprising, consisting essentially of, or consisting of as a first active component: a compound of formula (I), formula (Ia), formula (II), or formula (IIa) of the disclosure; and as a second active component selected from (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone; and a pharmaceutically acceptable excipient.

The invention also relates to methods of preventing or treating inflammation and/or pain, preventing or treating a neurological disorder, modulating activity of a mitogen activating protein (MAP), modulating neurogenesis, or modulating neurite outgrowth comprising the step of administering to a subject in need thereof a therapeutically effective amount of a compound of formula (I), formula (Ia), formula (II), or formula (IIa) and to administering a pharmaceutical composition or a composition according to the invention.

The invention also relates to 2-[4-(acetyloxy)-1H-indol-3-yl]ethyl}(methyl)azanium chloride (4-AcO-NMT chloride) and to its crystalline form. 4-AcO-NMT chloride has the following chemical formula:

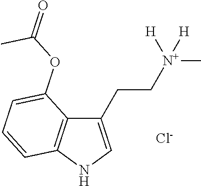 [SMILES: C[NH2+]CCc1c[nH]c2cccc(OC(C)=O)c12 ; [Cl-]]

The invention also relates to compositions comprising 4-AcO-NMT chloride or its crystalline form. The disclosure further relates to pharmaceutical compositions containing a therapeutically effective amount of 4-AcO-NMT chloride or its crystalline form and an excipient.

The invention relates to compositions comprising, consisting essentially of, or consisting of 4-AcO-NMT chloride or its crystalline form and an excipient. The invention also relates pharmaceutical compositions comprising a therapeutically effective amount of 4-AcO-NMT chloride or its crystalline form, wherein the excipient is a pharmaceutically acceptable carrier. The invention further relates to a method of preventing or treating a psychological disorder comprising the step of administering to a subject in need thereof a therapeutically effective amount of 4-AcO-NMT chloride or its crystalline form or of a pharmaceutical composition containing the compound.

The invention also relates to a composition comprising, consisting essentially of, or consisting of as a first active component: a compound of 4-AcO-NMT chloride or its crystalline form of the disclosure; and as a second active component selected from (a) a serotonergic drug, (b) a purified psilocybin derivative, (c) a purified cannabinoid and (d) a purified terpene, (e) an adrenergic drug, (f) a dopaminergic drug, (g) a monoamine oxidase inhibitor, (h) a purified erinacine, or (i) a purified hericenone; and a pharmaceutically acceptable excipient.

The invention also relates to methods of preventing or treating inflammation and/or pain, preventing or treating a neurological disorder, modulating activity of a mitogen activating protein (MAP), modulating neurogenesis, or modulating neurite outgrowth comprising the step of administering to a subject in need thereof a therapeutically effective amount of a compound of 4-AcO-NMT chloride or its crystalline form and to administering a pharmaceutical composition or a composition according to the invention.

### DESCRIPTION OF THE FIGURES

FIG. 1 shows the crystalline structure of 4-AcO-NMT chloride.

FIG. 2 shows a simulated x-ray powder diffraction pattern (XRPD) of crystalline 4-AcO-NMT chloride generated from its single crystal data.

### DETAILED DESCRIPTION

Compounds of the Invention

This invention relates to tryptamine compounds of formula (I):

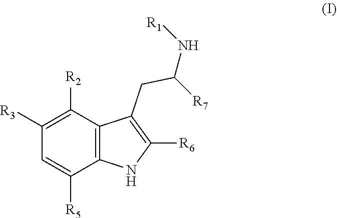

wherein
R₁ is a straight chain or branched C₁-C₆ alkyl or a straight chain or branched C₂-C₆ alkenyl;
R₂ and R₃ are independently chosen from hydrogen, hydroxyl, —OR₉, —OC(O)R₈, or —OC(O)OR₄, —OSO₂R₄;
R₄ is a straight chain or branched C₁-C₆ alkyl or a substituted or unsubstituted aryl;
R₈ is a straight chain or branched C₁-C₆ alkyl or a substituted or unsubstituted aryl;
R₉ is a straight chain or branched C₁-C₆ alkyl or a substituted or unsubstituted aryl; and
R₅, R₆ and R₇ are each independently hydrogen or a straight chain or branched C₁-C₆ alkyl; or a pharmaceutically acceptable acid-addition salt thereof.

In formula (I), R₁ is a straight chain or branched C₁-C₆ alkyl or a straight chain or branched C₂-C₆ alkenyl. R₁ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₁ may be a straight chain or branched C₁-C₄ alkyl, for example a straight chain C₁-C₄ alkyl, or a C₂-C₄ alkenyl. R₁ may be selected from straight chain or branched C₂-C₆ alkyl or C₃-C₆ alkyl. R₁ may be selected from methyl, ethyl, n-propyl, isopropyl, n-butyl, isobutyl, or tert-butyl. In other embodiments, R₁ may be methyl, ethyl, propyl, or isopropyl.

In formula (I), R₂ and R₃ are independently chosen from hydrogen, hydroxyl, —OR₉, —OC(O)R₈, —OC(O)OR₄, or —OSO₂R₄.

R₄, R₈, and R₉ are independently straight chain or branched C₁-C₆ alkyl or a substituted or unsubstituted aryl. When R₄, R₈, or R₉ is a straight chain or branched C₁-C₆ alkyl, it may be a straight chain or branched C₁-C₄ alkyl, for example a straight chain C₁-C₄ alkyl. R₄, R₈, and R₉ may be independently selected from straight chain or branched C₂-C₆ alkyl or C₃-C₆ alkyl. R₄, R₈, and R₉ may be independently a methyl, a tert-butyl, a phenyl or a para-tolyl group. In some embodiments, R₄, R₈, and R₉ may be independently methyl, ethyl, n-propyl or n-butyl, and for example may be methyl or ethyl. R₄, R₈, and R₉ may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more straight chain or branched C₁-C₄ alkyl groups, straight chain or branched C₁-C₄ hydroxyalkyl groups, hydroxyl groups or halo groups (e.g., F, Cl, I, or Br). When an aryl group is substituted with one or more straight chain or branched C₁-C₄ alkyl groups the group may be methyl, ethyl, n-propyl, isopropyl, n-butyl, isobutyl or tert-butyl or the group may be methyl, ethyl, isopropyl, or tert-butyl. In some embodiments, R₉ may be straight chain or branched C₃-C₅ alkyl. In other embodiments, R₈ may be straight chain or branched C₂-C₆ alkyl or C₇-C₁₄ aryl.

R₅, R₆ and R₇ in formula (I) are each independently hydrogen or a straight chain or branched C₁-C₆ alkyl, for example a straight chain C₁-C₆ alkyl. R₅, R₆ and R₇ may be each independently selected from straight chain or branched C₂-C₆ alkyl or C₃-C₆ alkyl. In some embodiments, R₅, R₆ and R₇ may be each independently selected from hydrogen, methyl, ethyl, n-propyl, isopropyl, n-butyl and isobutyl. In other embodiments, R₅, R₆, and R₇ may be independently hydrogen, methyl, or ethyl. In some embodiments, R₆ may be hydrogen or straight chain or branched C₃-C₆ alkyl. In other embodiments R₇ may be hydrogen or straight chain or branched C₂-C₆ alkyl.

Pharmaceutically acceptable salts of formula (I) may be any acid (e.g., HX or H₂X) addition salts. The anion, X⁻, may be any pharmaceutically acceptable anion, for example, Cl⁻, I⁻, Br⁻, ascorbate, or hydrofumarate, and the like. Other pharmaceutically acceptable salts may be prepared by anion exchange techniques known in the art to exchange the iodide anion for a desired pharmaceutically acceptable anion. For example, the iodide anion may be exchanged using an anion exchange resin.

Exemplary compounds of formula (I) are those wherein R₂ and R₃ are independently hydrogen or straight chain or branched —OR₉, —OC(O)OR₄, or —OSO₂R₄, wherein R₉ is straight chain or branched C₃-C₅ alkyl.

Other exemplary compounds of formula (I) are those where one of R₂ and R₃ is hydrogen and the other of R₂ and R₃ is —OC(O)OR₄ or —OSO₂R₄.

Other exemplary compounds of formula (I) are those where R₂ and R₃ are both hydrogen.

Other exemplary compounds of formula (I) are those where one of R₂ and R₃ is hydrogen and the other of R₂ and R₃ is —OC(O)OR₄.

Other exemplary compounds of formula (I) are those where one of R₂ and R₃ is hydrogen and the other of R₂ and R₃ is selected from —OR₉, wherein R₉ is straight chain or branched C₃-C₅ alkyl.

Other exemplary compounds of formula (I) are those where R₂ and R₃ are independently selected from hydrogen and —OC(O)R₈, wherein R₈ is selected from straight chain or branched C₂-C₆ alkyl. Still other exemplary compounds of formula (I) are those where one of R₂ and R₃ is hydrogen and the other of R₂ and R₃ is selected from —OC(O)R₈, wherein R₈ is selected from straight chain or branched C₂-C₆ alkyl.

Other exemplary compounds of formula (I) are those where R₁ is ethyl, straight chain or branched propyl, butyl, pentyl and hexyl, and wherein R₂ and R₃ are independently selected from hydrogen and —OC(O)R₈, wherein R₈ is straight chain or branched C₁-C₆ alkyl. Still other exemplary compounds of formula (I) are those where R₁ ethyl, straight chain or branched propyl, butyl, pentyl and hexyl, and where one of R₂ and R₃ is hydrogen and the other of R₂ and R₃ is selected from —OC(O)R₈, wherein R₈ is straight chain or branched C₁-C₆ alkyl.

Other exemplary compounds of formula (I) are those where R₂ and R₃ are independently selected from hydrogen and —OC(O)R₈, wherein R₈ is selected from substituted or unsubstituted C₇-C₁₄ aryl.

Other exemplary compounds of formula (I) are those where R₃ is hydrogen and R₂ is —OC(O)R₈, wherein R₈ is substituted or unsubstituted aryl.

Other exemplary compounds of formula (I) are those where R₁ is ethyl, straight chain or branched butyl, pentyl or hexyl, and wherein R₂ and R₃ are independently hydrogen or —OC(O)R₈, wherein R₈ is straight chain or branched C₁-C₆ alkyl. Still other exemplary compounds of formula (I) are those where R₁ is ethyl, straight chain or branched butyl, pentyl or hexyl, and wherein one of R₂ and R₃ is hydrogen and the other of R₂ and R₃ is —OC(O)R₈, wherein R₈ is straight chain or branched C₁-C₆ alkyl.

Other exemplary compounds of formula (I) are those where R₇ is hydrogen.

Other exemplary compounds of formula (I) are those where R₇ is straight chain or branched C₂-C₆ alkyl.

Other exemplary compounds of formula (I) are those where R₆ is straight chain or branched C₃-C₆ alkyl.

Other exemplary compounds of formula (I) are those where R₁ is methyl.

Other exemplary compounds of formula (I) are those where R₁ is ethyl.

Other exemplary compounds of formula (I) are those where R₁ is straight chain or branched propyl.

Other exemplary compounds of formula (I) are those where R₁ is isopropyl.

Other exemplary compounds of formula (I) are those where R₁ is straight chain or branched butyl.

Other exemplary compounds of formula (I) are those where R₁ is straight chain or branched pentyl.

Other exemplary compounds of formula (I) are those where R₁ is straight chain or branched hexyl.

This invention also relates to tryptamine compounds of formula (Ia):

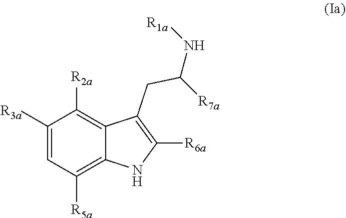

wherein
R₁ₐ is a straight chain or branched C₁-C₆ alkyl or a straight chain or branched C₂-C₆ alkenyl;
one of R₂ₐ and R₃ₐ is hydrogen and the other of R₂ and R₃ is selected from —OC(O)R₈ₐ, —OC(O)OR₄ₐ, and —OSO₂R₄ₐ;
R₄ₐ is a straight chain or branched C₁-C₆ alkyl or a substituted or unsubstituted aryl;
R₈ₐ is selected from straight chain or branched C₁-C₆ alkyl;
R₅ₐ, R₆ₐ and R₇ₐ are each independently hydrogen or a straight chain or branched C₁-C₆ alkyl; or a pharmaceutically acceptable acid-addition salt thereof.

In formula (Ia), R₁ₐ is a straight chain or branched C₁-C₆ alkyl or a straight chain or branched C₂-C₆ alkenyl. R₁ₐ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₁ₐ is selected from straight chain or branched C₂-C₆ alkyl or C₃-C₆ alkyl. In some embodiments, Ria may be a straight chain or branched C₁-C₄ alkyl, for example a straight chain C₁-C₄ alkyl, or a C₂-C₄ alkenyl. R₁ₐ may be selected from methyl, ethyl, n-propyl, isopropyl, n-butyl, isobutyl, or tert-butyl. In other embodiments, R₁ₐ may be methyl, ethyl, propyl, or isopropyl.

In formula (Ia), one of R₂ₐ and R₃ₐ is hydrogen and the other is —OC(O)R₈, —OC(O)OR₄ₐ, or —OSO₂R₄ₐ.

In formular (Ia), R₄ₐ is straight chain or branched C₁-C₆ alkyl or a substituted or unsubstituted aryl, and R₈ₐ is straight chain or branched C₁-C₆ alkyl. When R₄ₐ or R₈ₐ is a straight chain or branched C₁-C₆ alkyl, it may be a straight chain or branched C₁-C₄ alkyl, for example a straight chain C₁-C₄ alkyl. In some embodiments, R₄ₐ or R₈ₐ are independently selected from straight chain or branched C₂-C₆ alkyl or C₃-C₆ alkyl. R₄ₐ and R₈ₐ may be independently a methyl, a tert-butyl, a phenyl or a para-tolyl group. In some embodiments, R₄ₐ and R₈ₐ may be independently methyl, ethyl, n-propyl or n-butyl, and for example may be methyl or ethyl. R₄ₐ may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more straight chain or branched C₁-C₄ alkyl groups, straight chain or branched C₁-C₄ hydroxyalkyl groups, hydroxyl groups or halo groups (e.g. F, Cl, I, or Br). When an aryl group is substituted with one or more straight chain or branched C₁-C₄ alkyl groups the group may be methyl, ethyl, n-propyl, isopropyl, n-butyl, isobutyl or tert-butyl or the group may be methyl, ethyl, isopropyl, or tert-butyl. In some embodiments, R₈ₐ may be straight chain or branched C₂-C₆ alkyl or a straight chain or branched C₃-C₆ alkyl.

R₅ₐ, R₆ₐ and R₇ₐ in formula (Ia) are each independently hydrogen or a straight chain or branched C₁-C₆ alkyl, for example a straight chain C₁-C₆ alkyl. In some embodiments, R₄ₐ or R₈ₐ are independently selected from hydrogen and straight chain or branched C₂-C₆ alkyl or C₃-C₆ alkyl. In some embodiments, R₅ₐ, R₆ₐ, and R₇ₐ may be each independently selected hydrogen, methyl, ethyl, n-propyl, isopropyl, n-butyl and isobutyl. In other embodiments, R₅ₐ, R₆ₐ, and R₇ₐ may be independently hydrogen, methyl, or ethyl. In some embodiments, R₆ₐ may be hydrogen or straight chain or branched C₃-C₆ alkyl. In other embodiments R₇ₐ may be hydrogen or straight chain or branched C₂-C₆ alkyl.

Pharmaceutically acceptable salts of formula (Ia) may be acid (e.g., HX or H₂X) addition salts. The anion, X⁻, may be any pharmaceutically acceptable anion, for example, Cl⁻, I⁻, Br⁻, ascorbate, hydrofumarate, and the like. When the pharmaceutically acceptable anion is a di-anion it balances two of the ammonium cations. Other pharmaceutically acceptable salts may be prepared by anion exchange techniques known in the art to exchange the iodide anion for a desired pharmaceutically acceptable anion. For example, the iodide anion may be exchanged using an anion exchange resin.

Exemplary compounds of formula (Ia) are those where R₂ₐ is —OC(O)R₈.

Other exemplary compounds of formula (Ia) are those where R₂ₐ is —OC(O)OR₄ₐ.

Other exemplary compounds of formula (Ia) are those where R₂ₐ is —OSO₂R₄ₐ.

Other exemplary compounds of formula (Ia) are those where R₈ₐ is straight chain or branched C₂-C₆ alkyl.

Other exemplary compounds of formula (Ia) are those where R₈ₐ is straight chain or branched C₃-C₆ alkyl.

Other exemplary compounds of formula (Ia) are those where R₅ₐ is hydrogen.

Other exemplary compounds of formula (Ia) are those where R₆ₐ is hydrogen.

Other exemplary compounds of formula (Ia) are those where R₇ₐ is hydrogen.

Other exemplary compounds of formula (Ia) are those where R₁ₐ is methyl.

Other exemplary compounds of formula (Ia) are those where R₁ₐ is ethyl.

Other exemplary compounds of formula (Ia) are those where R₁ₐ is straight chain or branched propyl.

Other exemplary compounds of formula (Ia) are those where R₁ₐ is isopropyl.

Other exemplary compounds of formula (Ia) are those where R₁ₐ is straight chain or branched butyl.

Other exemplary compounds of formula (Ia) are those where R₁ₐ is straight chain or branched pentyl.

Other exemplary compounds of formula (Ia) are those where R₁ₐ is straight chain or branched hexyl.

This invention also relates to tryptamine compounds of formula (II):

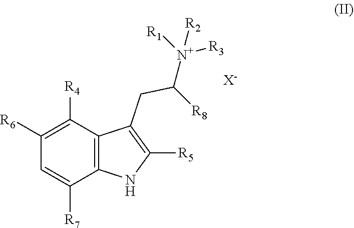

wherein
R₁ is a straight chain or branched C₁-C₆ alkyl or a straight chain or branched C₂-C₆ alkenyl;
R₂ and R₃ are both hydrogen;
R₄ and R₆ are independently chosen from hydrogen, hydroxyl, —OR₅, —OC(O)R₁₁, —OC(O)OR₁₂, —OSO₂R₁₂;
R₅ is a straight chain or branched C₁-C₆ alkyl or a substituted or unsubstituted aryl;
R₁₁ is a straight chain or branched C₁-C₆ alkyl or a substituted or unsubstituted aryl;
R₁₂ is a straight chain or branched C₁-C₆ alkyl or a substituted or unsubstituted aryl;
R₇, R₈ and R₉ are each independently hydrogen or a straight chain or branched C₁-C₆ alkyl; and
X⁻ is a pharmaceutically-acceptable anion.

In formula (II), R₁ is a straight chain or branched C₁-C₆ alkyl or a straight chain or branched C₂-C₆ alkenyl. R₁ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments,
R₁ may be a straight chain or branched C₁-C₄ alkyl, for example a straight chain C₁-C₄ alkyl, or a C₂-C₄ alkenyl. In some embodiments, R₁ selected from straight chain or branched C₂-C₆ alkyl or C₃-C₆ alkyl. R₁ may be selected from methyl, ethyl, n-propyl, isopropyl, n-butyl, isobutyl, or tert-butyl. In other embodiments, R₁ may be methyl, ethyl, propyl, or isopropyl.

In formula (II), R₂ and R₃ are both hydrogen.

In formula (II), R₄ and R₆ are independently chosen from hydrogen, hydroxyl, —OR₅, —OC(O)R₁₁, —OC(O)OR₁₂, or —OSO₂R₁₂.

R₅, R₁₁, and R₁₂ are independently straight chain or branched C₁-C₆ alkyl or a substituted or unsubstituted aryl. In some embodiments, R₅, R₁₁, and R₁₂ are independently selected from straight chain or branched C₂-C₆ alkyl or C₃-C₆ alkyl. When R₅, R₁₁, or R₁₂ is a straight chain or branched C₁-C₆ alkyl, it may be a straight chain or branched C₁-C₄ alkyl, for example a straight chain C₁-C₄ alkyl. R₅, R₁₁, and R₁₂ may be independently a methyl, a tert-butyl, a phenyl or a para-tolyl group. In some embodiments, R₅, R₁₁, and R₁₂ may be independently methyl, ethyl, n-propyl or n-butyl, and for example may be methyl or ethyl. R₅, R₁₁, and R₁₂ may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more straight chain or branched C₁-C₄ alkyl groups, straight chain or branched C₁-C₄ hydroxyalkyl groups, hydroxyl groups or halo groups (e.g., F, Cl, I, or Br). When an aryl group is substituted with one or more straight chain or branched C₁-C₄ alkyl groups the group may be methyl, ethyl, n-propyl, isopropyl, n-butyl, isobutyl or tert-butyl or the group may be methyl, ethyl, isopropyl, or tert-butyl. In some embodiments, R₅ may be straight chain or branched C₃-C₅ alkyl. In other embodiments, R₁₁ may be straight chain or branched C₂-C₆ alkyl.

R₇, R₈, and R₉ in formula (II) are each independently hydrogen or a straight chain or branched C₁-C₆ alkyl, for example a straight chain C₁-C₆ alkyl. In some embodiments, R₅, R₆ and R₇ may be each independently selected hydrogen, methyl, ethyl, n-propyl, isopropyl, n-butyl and isobutyl. In other embodiments, R₅, R₆, and R₇ may be independently hydrogen, methyl, or ethyl. In some embodiments, R₇ may be straight chain or branched C₂-C₆ alkyl.

Pharmaceutically acceptable anions of formula (II) may be any acid (e.g., HX or H₂X) addition anions. X⁻ may be Cl⁻, I⁻, Br⁻, ascorbate, hydrofumarate, and the like. When the pharmaceutically acceptable anion is a di-anion it balances two of the ammonium cations. Other pharmaceutically acceptable anions may be prepared by anion exchange techniques known in the art to exchange the iodide anion for a desired pharmaceutically acceptable anion. For example, the iodide anion may be exchanged using an anion exchange resin.

Exemplary compounds of formula (II) are those where one of R₄ and R₆ is hydrogen and the other of R₄ and R₆—OC(O)OR₄ or —OSO₂R₄.

Other exemplary compounds of formula (II) are those where one of R₄ and R₆ is hydrogen and the other of R₄ and R₆ is —OSO₂R₁₂.

Other exemplary compounds of formula (II) are those where one of R₄ and R₆ is hydrogen and the other of R₄ and R₆ is —OC(O)OR₁₂.

Other exemplary compounds of formula (II) are those where one of R₄ and R₆ is hydrogen and the other of R₄ and R₆ is —OR₅. Still other exemplary compounds of formula (II) are those where one of R₄ and R₆ is hydrogen and the other of R₄ and R₆ is —OR₅, where R₅ is selected from straight chain or branched C₃-C₅ alkyl

Other exemplary compounds of formula (II) are those where R₄ and R₆ are independently hydrogen or —OC(O)R₁₁. Still other exemplary compounds of formula (II) are those where R₄ and R₆ are independently selected from hydrogen and —OC(O)R₁₁, where R₁₁ is selected from straight chain or branched C₂-C₆ alkyl.

Other exemplary compounds of formula (II) are those where R₁ is ethyl, straight chain or branched propyl, butyl, pentyl or hexyl, and where R₄ and R₆ are independently hydrogen or —OC(O)R₁₁, where R₁₁ is a straight chain or branched C₁-C₆ alkyl.

Other exemplary compounds of formula (II) are those where R₁ is ethyl, straight chain or branched propyl, butyl, pentyl or hexyl, and where one of R₄ and R₆ is hydrogen and the other of R₄ and R₆ is —OC(O)R₁₁, where R₁₁ is a straight chain or branched C₁-C₆ alkyl.

Other exemplary compounds of formula (II) are those where R₁ is ethyl, straight chain or branched butyl, pentyl, or hexyl, and where R₄ and R₆ are independently hydrogen or —OC(O)R₁₁, where R₁₁ is straight chain or branched C₁-C₆ alkyl.

Other exemplary compounds of formula (II) are those where R₁ is selected from ethyl, straight chain or branched butyl, pentyl, or hexyl, and where of R₄ and R₆ is hydrogen and the other of R₄ and R₆ is —OC(O)R₁₁, where R₁₁ is straight chain or branched C₁-C₆ alkyl.

Other exemplary compounds of formula (II) are those where R₇ is hydrogen

Other exemplary compounds of formula (II) are those where R₇ is methyl.

Other exemplary compounds of formula (II) are those where R₇ is straight chain or branched C₂-C₆ alkyl.

Other exemplary compounds of formula (II) are those where R₈ is hydrogen.

Other exemplary compounds of formula (II) are those where R₈ is methyl.

Other exemplary compounds of formula (II) are those where R₈ is ethyl.

Other exemplary compounds of formula (II) are those where R₉ is hydrogen.

Other exemplary compounds of formula (II) are those where R₁ is methyl.

Other exemplary compounds of formula (II) are those where R₁ is ethyl.

Other exemplary compounds of formula (II) are those where R₁ is straight chain or branched propyl.

Other exemplary compounds of formula (II) are those where R₁ is isopropyl.

Other exemplary compounds of formula (II) are those where R₁ is straight chain or branched butyl.

Other exemplary compounds of formula (II) are those where R₁ is straight chain or branched pentyl.

Other exemplary compounds of formula (II) are those where R₁ is straight chain or branched hexyl.

Other exemplary compounds of formula (II) are those where X⁻ comprises a hydrofumarate anion.

Other exemplary compounds of formula (II) are those with the proviso that X⁻ is not chloride when R₁ is methyl, R₄ is —OC(O)R₁₁, R₁₁ is methyl, and R₆, R₇, R₈, and R₉ are hydrogen.

An exemplary compound of formula of formula (II) is that where R₁ is methyl, R₂ and R₃ are both hydrogen, R₄ is —OC(O)R₁₁, R₆-R₉ are hydrogen, R₁₁ is methyl, and X⁻ is chloride: 2-[4-(acetyloxy)-1H-indol-3-yl]ethyl}(methyl)azanium chloride (4-AcO-NMT chloride). 4-AcO-NMT chloride has the following chemical formula:

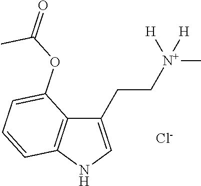 [SMILES: C[NH2+]CCc1c[nH]c2cccc(OC(C)=O)c12 ; [Cl-]]

Another exemplary compound of formula (II) is that where R₁ is methyl, R₂ and R₃ are both hydrogen, R₄ is a hydroxyl, R₆-R₉ are hydrogen, and X⁻ is hydrofumarate: 4-hydroxy-N-methyltryptammonium hydrofumarate (4-HO-NMT hydrofumarate). 4-HO-NMT hydrofumarate has the following chemical formula:

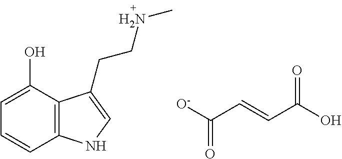

This invention also relates to tryptamine compounds of formula (IIa):

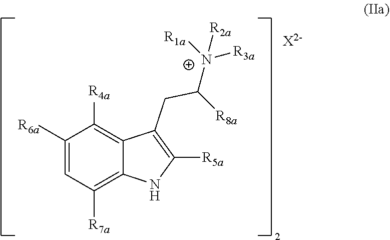

wherein
R₁ₐ is a straight chain or branched C₁-C₆ alkyl or a straight chain or branched C₂-C₆ alkenyl;
R₂ₐ and R₃ₐ are both hydrogen;
R₄ₐ and R₆ₐ are independently chosen from hydrogen, hydroxyl, —OR₅ₐ, —OC(O)R₁₁ₐ, —OC(O)OR₁₂ₐ, —OSO₂R₁₂ₐ;
R₅ₐ is a straight chain or branched C₁-C₆ alkyl or a substituted or unsubstituted aryl;
R₁₁ₐ is a straight chain or branched C₁-C₆ alkyl or a substituted or unsubstituted aryl;
R₁₂ₐ is a straight chain or branched C₁-C₆ alkyl or a substituted or unsubstituted aryl;
R₇ₐ, R₈ₐ and R₉ₐ are each independently hydrogen or a straight chain or branched C₁-C₆ alkyl; and
X²⁻ is a pharmaceutically-acceptable dianion.

In formula (IIa), R₁ₐ is a straight chain or branched C₁-C₆ alkyl or a straight chain or branched C₂-C₆ alkenyl. In some embodiments, R₁ₐ selected from straight chain or branched C₂-C₆ alkyl or C₃-C₆ alkyl. R₁ₐ may be a straight chain or branched C₁-C₆ alkyl, for example a straight chain C₁-C₆ alkyl, or a straight chain or branched C₂-C₆ alkenyl, for example vinyl, allyl, 2-butenyl, etc. In some embodiments, R₁ may be a straight chain or branched C₁-C₄ alkyl, for example a straight chain C₁-C₄ alkyl, or a C₂-C₄ alkenyl. R₁ₐ may be selected from methyl, ethyl, n-propyl, isopropyl, n-butyl, isobutyl, or tert-butyl. In other embodiments, R₁ₐ may be methyl, ethyl, propyl, or isopropyl.

In formula (II), R₂ₐ and R₃ₐ are both hydrogen.

In formula (II), R₄ₐ and R₆ₐ are independently chosen from hydrogen, hydroxyl, —OR₅, —OC(O)R₁₁, —OC(O)OR₁₂, or —OSO₂R₁₂.

R₅ₐ, R₁₁ₐ, and R₁₂ₐ are independently straight chain or branched C₁-C₆ alkyl or a substituted or unsubstituted aryl. In some embodiments, R₅ₐ, R₁₁ₐ, and R₁₂ₐ are independently selected from straight chain or branched C₂-C₆ alkyl or C₃-C₆ alkyl. When R₅ₐ, R₁₁ₐ, or R₁₂ₐ is a straight chain or branched C₁-C₆ alkyl, it may be a straight chain or branched C₁-C₄ alkyl, for example a straight chain C₁-C₄ alkyl. R₅ₐ, R₁₁ₐ, and R₁₂ₐ may be independently a methyl, a tert-butyl, a phenyl or a para-tolyl group. In some embodiments, R₅ₐ, R₁₁ₐ, and R₁₂ₐ may be independently methyl, ethyl, n-propyl or n-butyl, and for example may be methyl or ethyl. R₅ₐ, R₁₁ₐ, and R₂ₐ may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more straight chain or branched C₁-C₄ alkyl groups, straight chain or branched C₁-C₄ hydroxyalkyl groups, hydroxyl groups or halo groups (e.g., F, Cl, I, or Br). When an aryl group is substituted with one or more straight chain or branched C₁-C₄ alkyl groups the group may be methyl, ethyl, n-propyl, isopropyl, n-butyl, isobutyl or tert-butyl or the group may be methyl, ethyl, isopropyl, or tert-butyl. In some embodiments, R₅ₐ may be straight chain or branched C₃-C₅ alkyl or straight chain or branched hexyl. In other embodiments, R₁₁ₐ may be straight chain or branched C₂-C₆ alkyl.

R₇ₐ, R₈ₐ, and R₉ₐ in formula (IIa) are each independently hydrogen or a straight chain or branched C₁-C₆ alkyl, for example a straight chain C₁-C₆ alkyl. In some embodiments, R₇ₐ, R₈ₐ, and R₉ₐ are independently selected from hydrogen and straight chain or branched C₂-C₆ alkyl or C₃-C₆ alkyl. In some embodiments, R₅ₐ, R₆ₐ and R₇ₐ may be each independently selected hydrogen, methyl, ethyl, n-propyl, isopropyl, n-butyl and isobutyl. In other embodiments, R₅ₐ, R₆ₐ, and R₇ₐ may be independently hydrogen, methyl, or ethyl. In some embodiments, R₇ₐ may be straight chain or branched C₂-C₆ alkyl.

Pharmaceutically acceptable dianions of formula (IIa) may be acid (e.g., H₂X) addition dianions. X²⁻ may be fumarate, malonate, succinate, tartarate, oxalate, and maleate, and the like. When the pharmaceutically acceptable anion is a dianion it balances two of the ammonium cations. Other pharmaceutically acceptable anions may be prepared by anion exchange techniques known in the art to exchange the iodide anion for a desired pharmaceutically acceptable anion. For example, the iodide anion may be exchanged using an anion exchange resin.

Exemplary compounds of formula (IIa) are those where one of R₄ₐ and R₆ₐ is hydrogen and the other of R₄ₐ and R₆ₐ is —OC(O)OR₄ or —OSO₂R₄.

Exemplary compounds of formula (IIa) are those where one of R₄ₐ and R₆ₐ is hydrogen and the other of R₄ₐ and R₆ₐ is —OSO₂R₁₂.

Exemplary compounds of formula (IIa) are those where one of R₄ₐ and R₆ₐ is hydrogen and the other of R₄ₐ and R₆ₐ is —OC(O)OR₁₂.

Exemplary compounds of formula (IIa) are those where one of R₄ₐ and R₆ₐ is hydrogen and the other of R₄ₐ and R₆ₐ is —OR₅. Still other exemplary compounds of formula (IIa) are those where one of R₂and R₃ is hydrogen and the other of R₄ₐ and R₆ₐ is —OR₅, where R₅ is methyl, ethyl, straight chain or branched C₃-C₅ alkyl, or straight chain or branched hexyl.

Exemplary compounds of formula (IIa) are those where R₂ₐ and R₃ₐ are independently hydrogen or —OC(O)R₁₁.

Exemplary compounds of formula (IIa) are those where one of R₄ₐ and R₆ₐ is hydrogen and the other of R₄ₐ and R₆ₐ is —OC(O)R₁₁. Still other exemplary compounds of formula (IIa) are those where one of R₄ₐ and R₆ₐ is hydrogen and the other of R₄ₐ and R₆ₐ is —OC(O)R₁₁ₐ, where R₁₁ₐ is methyl or straight chain or branched C₂-C₆ alkyl.

Exemplary compounds of formula (IIa) are those where R₁ₐ is ethyl, straight chain or branched propyl, butyl, pentyl or hexyl, and where R₄ₐ and R₆ₐ are independently hydrogen or —OC(O)R₁₁ₐ, where R₁₁ₐ is straight chain or branched C₁-C₆ alkyl.

Exemplary compounds of formula (IIa) are those where R₁ₐ is ethyl, straight chain or branched propyl, butyl, pentyl or hexyl, and where one of R₄ₐ and R₆ₐ is hydrogen and the other of R₄ₐ and R₆ₐ is selected from —OC(O)R₁₁ₐ, where R₁₁ₐ is straight chain or branched C₁-C₆ alkyl.

Exemplary compounds of formula (IIa) are those where R₁ₐ is ethyl, straight chain or branched butyl, pentyl or hexyl, and where R₄ₐ and R₆ₐ are independently hydrogen or —OC(O)R₁₁ₐ, where R₁₁ₐ is selected from straight chain or branched C₁-C₆ alkyl.

Exemplary compounds of formula (IIa) are those where R₁ₐ is ethyl, straight chain or branched butyl, pentyl or hexyl, and where of R₄ₐ and R₆ₐ is hydrogen and the other of R₄ₐ and R₆ₐ is —OC(O)R₁₁ₐ, where R₁₁ₐ is straight chain or branched C₁-C₆ alkyl.

Exemplary compounds of formula (IIa) are those where R₇ₐ is hydrogen.

Exemplary compounds of formula (IIa) are those where R₇ₐ is methyl.

Exemplary compounds of formula (IIa) are those where R₇ₐ is straight chain or branched C₂-C₆ alkyl.

Exemplary compounds of formula (IIa) are those where R₈ₐ is hydrogen.

Exemplary compounds of formula (IIa) are those where R₈ₐ is methyl.

Exemplary compounds of formula (IIa) are those where R₈ₐ is ethyl.

Exemplary compounds of formula (IIa) are those where R₉ₐ is hydrogen.

Exemplary compounds of formula (IIa) are those where R₁ₐ is methyl.

Exemplary compounds of formula (IIa) are those where R₁ₐ is ethyl.

Exemplary compounds of formula (IIa) are those where R₁ₐ is straight chain or branched propyl.

Exemplary compounds of formula (IIa) are those where R₁ₐ is isopropyl.

Exemplary compounds of formula (IIa) are those where R₁ₐ is straight chain or branched butyl.

Exemplary compounds of formula (IIa) are those where R₁ₐ is straight chain or branched pentyl.

Exemplary compounds of formula (IIa) are those where R₁ₐ is straight chain or branched hexyl.

Exemplary compounds of formula (IIa) are those where X²⁻ comprises a fumarate dianion.

Exemplary compounds of formula (IIa) are those with the proviso that X² is not a fumarate dianion when R₁ₐ is methyl, R₄ₐ is hydroxyl, and R₆ₐ, R₇ₐ, R₈ₐ, and R₉ₐ are hydrogen.

An exemplary compound of formula (IIa) is that where R₁ₐ is methyl, R₂ₐ and R₃ₐ are both hydrogen, R₄ₐ is —OCOR₁₁ₐ, R₆ₐ-R₉ₐ are hydrogen, R₁₁ₐ is methyl, and X² is fumarate: 4-acetoxy-N-methyltryptammonium fumarate (4-AcO-NMT fumarate). 4-AcO-NMT fumarate has the following chemical formula:

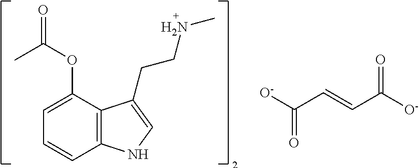

A compound of formula (I), formula (Ia), formula (II), or formula (IIa) may be prepared by a variety of methods known in the organic synthesis art. In some cases, mono-substituted amines of formula (I), formula (Ia), formula (II), or formula (IIa) may be prepared by treating a primary amine with an alkyl halide or an alkenyl halide (e.g. vinyl bromide) in a suitable solvent to provide a monoalkylated product. In some cases, monoalkylation may be accomplished via reductive amination of the corresponding amine (R₁/R₁ₐ═H) with an appropriate aldehyde. For example, when R₁/R₁ₐ is methyl, reductive amination of the primary amine using formaldehyde; when R₁/R₁ₐ is ethyl, acetaldehyde or when R₁/R₁ₐ is propyl, propyl aldehyde. Other methods for synthesizing amines can be found in textbooks known in the art, for example, Smith, M. B., March's Advanced Organic Chemistry: Reactions, Mechanisms, and Structure, Wiley; 8th Edition (Feb. 19, 2020).

The invention also relates to 4-AcO-NMT chloride and to its crystalline form.

Methods of Treatment and Therapeutic Uses

Compounds of formula (I), (Ia), (II), or (IIa) according to the disclosure, crystalline forms thereof, and the methods and the compositions (e.g., pharmaceutical compositions) are used to regulate the activity of a neurotransmitter receptor by administering a therapeutically effective dose of compounds of formula (I), (Ia), (II), or (IIa) according to the disclosure, and the methods and the compositions (e.g., pharmaceutical compositions) are used to treat inflammation and/or pain by administering a therapeutically effective dose of compounds of formula (I), (Ia), (II), or (IIa) according to the disclosure.

Methods of the disclosure also related to the administration of a therapeutically effective amount of compounds of formula (I), (Ia), (II), or (IIa) according to the disclosure to prevent or treat a disease or condition, such as those discussed below for a subject in need of treatment. Compounds of formula (I), (Ia), (II), or (IIa) according to the disclosure may be administered neat or as a composition comprising compounds of formula (I), (Ia), (II), or (IIa) according to the disclosure as discussed below.

Compounds of formula (I), (Ia), (II), or (IIa) according to the disclosure may be used to prevent and/or treat a psychological disorder. The disclosure provides a method for preventing and/or treating a psychological disorder by administering to a subject in need thereof a therapeutically effective amount of compounds of formula (I), (Ia), (II), or (IIa) according to the disclosure, including the exemplary embodiments discussed herein. The psychological disorder may be chosen from depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome, post-traumatic stress disorder (PTSD), premenstrual dysphoric disorder (PMDD), and premenstrual syndrome (PMS).

Compounds of formula (I), (Ia), (II), or (IIa) according to the disclosure may be used to prevent and/or treat a brain disorder. The disclosure provides a method for preventing and/or treating a brain disorder (e.g., Huntington's disease, Alzheimer's disease, dementia, and Parkinson's disease) by administering to a subject in need thereof a therapeutically effective amount of compounds of formula (I), (Ia), (II), or (IIa) according to the disclosure, including the exemplary embodiments discussed above.

Compounds of formula (I), (Ia), (II), or (IIa) according to the disclosure may be used to prevent and/or treat developmental disorders, delirium, dementia, amnestic disorders and other cognitive disorders, psychiatric disorders due to a somatic condition, drug-related disorders, schizophrenia and other psychotic disorders, mood disorders, anxiety disorders, somatoform disorders, factitious disorders, dissociative disorders, eating disorders, sleep disorders, impulse control disorders, adjustment disorders, or personality disorders. The disclosure provides a method for preventing and/or treating these disorders by administering to a subject in need thereof a therapeutically effective amount of compounds of formula (I), (Ia), (II), or (IIa) according to the disclosure, including the exemplary embodiments discussed above.

Compounds of formula (I), (Ia), (II), or (IIa) according to the disclosure may be used to prevent and/or treat inflammation and/or pain, such as for example inflammation and/or pain associated with inflammatory skeletal or muscular diseases or conditions. The disclosure provides a method for preventing and/or treating an inflammation and/or pain by administering to a subject in need thereof a therapeutically effective amount of compounds of formula (I), (Ia), (II), or (IIa) according to the disclosure, including the exemplary embodiments discussed herein. Generally speaking, treatable “pain” includes nociceptive, neuropathic, and mix-type. A method of the disclosure may reduce or alleviate the symptoms associated with inflammation, including but not limited to treating localized manifestation of inflammation characterized by acute or chronic swelling, pain, redness, increased temperature, or loss of function in some cases. A method of the disclosure may reduce or alleviate the symptoms of pain regardless of the cause of the pain, including but not limited to reducing pain of varying severity, i.e., mild, moderate and severe pain, acute pain and chronic pain. A method of the disclosure is effective in treating joint pain, muscle pain, tendon pain, burn pain, and pain caused by inflammation such as rheumatoid arthritis. Skeletal or muscular diseases or conditions which may be treated include but are not limited to musculoskeletal sprains, musculoskeletal strains, tendinopathy, peripheral radiculopathy, osteoarthritis, joint degenerative disease, polymyalgia rheumatica, juvenile arthritis, gout, ankylosing spondylitis, psoriatic arthritis, systemic lupus erythematosus, costochondritis, tendonitis, bursitis, such as the common lateral epicondylitis (tennis elbow), medial epicondylitis (pitchers elbow) and trochanteric bursitis, temporomandibular joint syndrome, and fibromyalgia.

Compounds of formula (I), (Ia), (II), or (IIa) according to the disclosure may be used to modulate activity of a mitogen activating protein (MAP), comprising administering a composition of the invention. In one embodiment, the mitogen activating protein (MAP) comprises a MAP kinase (MAPk). MAPKs provide a wide-ranging signaling cascade that allow cells to quickly respond to biotic and abiotic stimuli. Exemplary MAPKs include, but are not limited to, Tropomyosin Receptor Kinase A (TrkA), P38-alpha, Janus Kinase 1 (JAK1), and c-Jun N-Terminal Kinase 3 (JNK3). TrkA is a high affinity catalytic receptor of nerve growth factor (NGF) protein. TrkA regulates NGF response, influencing neuronal differentiation and outgrowth as well as programmed cell death. p38-alpha is involved with the regulation of pro-inflammatory cytokines, including TNF-α. In the central nervous system, p38-alpha regulates neuronal death and neurite degeneration, and it is a common target of Alzheimer's disease therapies. JAK1 influences cytokine signaling, including IL-2, IL-4, IFN-alpha/beta, IFN-γ, and IL-10, and it is implicated in brain aging. JNK3 is neuronal specific protein isoform of the JNKs. It is involved with the regulation of apoptosis. JNK3 also plays a role in modulating the response of cytokines, growth factors, and oxidative stress.

As used herein, the term “modulating activity of a mitogen activating protein” refers to changing, manipulating, and/or adjusting the activity of a mitogen activating protein. In one embodiment, modulating the activity of a MAP, such as a MAPK, can influence neural health, neurogenesis, neural growth and differentiation, and neurodegenerative diseases.

Compounds of formula (I), (Ia), (II), or (IIa) according to the disclosure may be used to modulate neurogenesis, comprising administering a composition of the invention. As used herein, the term “modulating neurite outgrowth” refers to changing, manipulating, and/or adjusting the growth and development of neural projections, or “neurites.” In one embodiment, neurogenesis comprises modulating the growth of new neurites, the number of neurites per neuron, and/or neurite length. In one embodiment, modulating neurite outgrowth comprises increasing and/or enhancing the rate and/or length at which neurites develop.

Compounds of formula (I), (Ia), (II), or (IIa) according to the disclosure may be used to modulate neurite outgrowth, comprising administering a composition of the invention. As used herein, the term “modulating neurogenesis” refers to changing, manipulating, and/or adjusting the growth and development of neural tissue. In one embodiment, neurogenesis comprises adult neurogenesis, in which new neural stem cells are generated from neural stem cells in an adult animal. In one embodiment, modulating neurogenesis comprises increasing and/or enhancing the rate at which new neural tissue is developed.

Compositions

The disclosure also relates to compositions comprising an effective amount of a compound of formula (I), (Ia), (II), or (IIa) according to the disclosure (monoalkyl tryptamine compounds of the disclosure), including its exemplary embodiments discussed above, and an excipient (e.g., a pharmaceutically-acceptable excipient). In another embodiment, the disclosure also relates to pharmaceutical compositions comprising a therapeutically effective amount of monoalkyl tryptamine compounds of the disclosure, including their exemplary embodiments discussed above, and a pharmaceutically acceptable excipient (also known as a pharmaceutically acceptable carrier). As discussed above, a monoalkyl tryptamine compound of the disclosure may be, for example, therapeutically useful to prevent and/or treat the psychological disorders, brain disorders, pain, and inflammation as well as the other disorders described herein.

A composition or a pharmaceutical composition of the disclosure may be in any form which contains a monoalkyl tryptamine compound of the disclosure. The composition may be, for example, a tablet, capsule, liquid suspension, injectable, topical, or transdermal. The compositions generally contain, for example, about 1% to about 99% by weight of a monoalkyl tryptamine compound of the disclosure and, for example, 99% to 1% by weight of at least one suitable pharmaceutically acceptable excipient. In one embodiment, the composition may be between about 5% and about 75% by weight of a monoalkyl tryptamine compound of the disclosure, with the rest being at least one suitable pharmaceutically acceptable excipient or at least one other adjuvant, as discussed below.

Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. Various ratios of these components in the composition are also disclosed. The disclosures of US 2018/0221396 A1 and US 2019/0142851 A1 are incorporated herein by reference. According to this disclosure, a monoalkyl tryptamine compound of the disclosure may be used as the “first purified psilocybin derivative” in the compositions described in US 2018/0221396 A1 and US 2019/0142851 A1. Accordingly, this disclosure provides a composition comprising: a first component comprising at least one monoalkyl tryptamine compound of the disclosure; at least one second component selected from at least one of (a) a serotonergic drug, (b) a purified psilocybin derivative, (c) a purified cannabinoid or (d) a purified terpene; and at least one pharmaceutically-acceptable excipient or at least one other adjuvant. Such a composition may be a pharmaceutical composition wherein the components are present individually in therapeutically effective amounts or by combination in a therapeutically effective amount to treat a disease, disorder, or condition as described herein.

When used in such compositions as a first component comprising at least one monoalkyl tryptamine compound of the disclosure with a second component selected from at least one of (a) a serotonergic drug, (b) a purified psilocybin derivative, (c) a purified cannabinoid, or (d) a purified terpene, the compositions represent particular embodiments of the invention. Compositions having as a first component at least one monoalkyl tryptamine compound of the disclosure with a second component selected from at least one of (e) an adrenergic drug, (f) a dopaminergic drug, (g) a monoamine oxidase inhibitor, (h) a purified erinacine, or (i) a purified hericenone, also represent additional particular embodiments of the invention represented by the compositions having the monoalkyl tryptamine compound of the disclosure. In some embodiments, the first and second components can be administered at the same time (e.g., together in the same composition), or at separate times over the course of treating a patient in need thereof. Such a composition may be a pharmaceutical composition wherein the components are present individually in therapeutically effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments, incorporated here by reference. Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed exemplary embodiments. Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments. Exemplary terpenes include but are not limited to the terpenes described in paragraphs [0160]-[0238] of US 2018/0221396 A1 and [0161]-[0300] US 2019/0142851 A1 as well as the disclosed exemplary embodiments.

A pharmaceutical formulation of the disclosure may comprise, consist essentially of, or consist of (a) at least one monoalkyl tryptamine compound of the disclosure and (b) at least one second active compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and (c) a pharmaceutically acceptable excipient. In some embodiments, the monoalkyl tryptamine compound(s) of the disclosure and the second active compound(s) are each present in a therapeutically effective amount using a purposefully engineered and unnaturally occurring molar ratios. Exemplary molar ratios of the monoalkyl tryptamine compounds of the disclosure to the second active compound in a composition of the disclosure include but are not limited to from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

A pharmaceutical formulation of the disclosure may comprise a composition containing a monoalkyl tryptamine compound of the disclosure and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a purified psilocybin derivative with a second purified psilocybin derivative, with one or two purified cannabinoids or with a purified terpene. The disclosures of US 2018/0221396 A1 and US 2019/0142851 A1 are incorporated herein by reference. According to this disclosure composition containing a monoalkyl tryptamine compound of the disclosure may be used in place of a “purified psilocybin derivative” in the compositions described in US 2018/0221396 A1 and US 2019/0142851 A1. Accordingly, the disclosure provides a pharmaceutical formulation comprising as (a) at least one monoalkyl tryptamine compound of the disclosure and at least one second component selected from (b) a purified psilocybin derivative, (c) a purified cannabinoid or (d) a purified terpene; and at least one pharmaceutically-acceptable excipient or at least one other adjuvant, as described herein. Such a composition may be a pharmaceutical composition wherein the components are present individually in therapeutic effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments, incorporated here by reference. Some exemplary serotonergic drugs include SSRIs and SNRIs. Some examples of specific serotonergic drugs include the following molecules, including any salts, solvates, or polymorphs thereof: 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2, alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N,N-dimethyl-T, N-Isopropyl-4-methoxy-N-methyl-T, N-Isopropyl-5-methoxy-N-methyl-T, 5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, and 7-Methoxy-1-methyl-1,2,3,4-tetrahydro-C, alpha,N-Dimethyl-5-methoxy-T. For additional information regarding these compounds see Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof. In an exemplary embodiment, the serotonergic drug is 3,4-methylenedioxymethamphetamine.

Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed exemplary embodiments, incorporated here by reference. In one embodiment, the compositions disclosed herein comprise one or more purified psilocybin derivatives chosen from: [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl]dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments, incorporated here by reference. Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (A8-THC), Delta-8-tetrahydrocannabinolic acid (A8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-metha- no-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed exemplary embodiments, incorporated here by reference. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, caryophyllene, caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, caryophyllene, cedrene, cymene, elemene, eucalyptol, eudesmol, farnesene, fenchol, geraniol, guaiacol, humulene, isoborneol, limonene, linalool, menthol, myrcene, nerolidol, ocimene, phellandrene, phytol, pinene, pulegone, sabinene, terpineol, terpinolene, or valencene.

As used herein, the term “adrenergic drug” refers to a compound that binds, blocks, or otherwise influences (e.g., via an allosteric reaction) activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.).

In one embodiment, an adrenergic drug is an antidepressant. In one embodiment, an adrenergic drug is a norepinephrine transporter inhibitor. In one embodiment, an adrenergic drug is a vesicular monoamine transporter inhibitor. In one embodiment, an adrenergic drug is chosen from adrenaline, agmatine, amoxapine, aptazapine, atomoxetine, bupropion, clonidine, doxepin, duloxetine, esmirtazpine, mianserin, ketanserin, mirabegron, mirtazapine, norepinephrine, phentolamine, phenylephrine, piperoxan, reserpine, ritodrine, setiptiline, tesofensine, timolol, trazodone, trimipramine, or xylazine.

As used herein, the term “dopaminergic drug” refers to a compound that binds, blocks, or otherwise influences (e.g., via an allosteric reaction) activity at a dopamine receptor. In one embodiment, a dopaminergic drug binds to a dopamine receptor. In one embodiment, a dopaminergic drug indirectly affects a dopamine receptor, e.g., via interactions affecting the reactivity of other molecules at the dopamine receptor. In one embodiment, a dopaminergic drug is an agonist, e.g., a compound activating a dopamine receptor. In one embodiment, a dopaminergic drug is an antagonist, e.g., a compound binding but not activating a dopamine receptor, e.g., blocking a receptor. In one embodiment, a dopaminergic drug is an effector molecule, e.g., a compound binding to an enzyme for allosteric regulation. In one embodiment, a dopaminergic drug acts (either directly or indirectly) at more than one type of receptor (e.g., 5HT, dopamine, adrenergic, acetylcholine, etc.).

In one embodiment, a dopaminergic drug is a dopamine transporter inhibitor. In one embodiment, a dopaminergic drug is a vesicular monoamine transporter inhibitor. In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, or thioridazine.

As used herein, the term “monoamine oxidase inhibitor” (MAOI) refers to a compound that blocks the actions of monoamine oxidase enzymes. In on embodiment, a MAOI inhibits the activity of one or both monoamine oxidase A and monoamine oxidase B. In one embodiment a MAOI is a reversible inhibitors of monoamine oxidase A. In one embodiment a MAOI is a drug chosen from isocarboxazid, phenelzine, or tranylcypromine.

In one embodiment, the compositions and methods disclosed herein include one or more purified erinacine molecules. In one embodiment, the compositions and methods disclosed herein comprise purified erinacine A. In one embodiment, the compositions and methods disclosed herein comprise erinacine B. In one embodiment, the compositions and methods disclosed herein comprise erinacine C. In one embodiment, the compositions and methods disclosed herein comprise erinacine D.

In one embodiment, the compositions and methods disclosed herein comprise erinacine E. In one embodiment, the compositions and methods disclosed herein comprise erinacine F. In one embodiment, the compositions and methods disclosed herein comprise erinacine G. In one embodiment, the compositions and methods disclosed herein comprise erinacine H. In one embodiment, the compositions and methods disclosed herein comprise erinacine I. In one embodiment, the compositions and methods disclosed herein comprise erinacine J. In one embodiment, the compositions and methods disclosed herein comprise erinacine K In one embodiment, the compositions and methods disclosed herein comprise erinacine P. In one embodiment, the compositions and methods disclosed herein comprise erinacine Q. In one embodiment, the compositions and methods disclosed herein comprise erinacine R. In one embodiment, the compositions and methods disclosed herein comprise erinacine S.

In one embodiment, the compositions and methods disclosed herein include one or more purified hericenone molecules. In one embodiment, the compositions and methods disclosed herein comprise purified hericenone A. In one embodiment, the compositions and methods disclosed herein comprise purified hericenone B. In one embodiment, the compositions and methods disclosed herein comprise purified hericenone C. In one embodiment, the compositions and methods disclosed herein comprise purified hericenone D. In one embodiment, the compositions and methods disclosed herein comprise purified hericenone E. In one embodiment, the compositions and methods disclosed herein comprise purified hericenone F. In one embodiment, the compositions and methods disclosed herein comprise purified hericenone G. In one embodiment, the compositions and methods disclosed herein comprise purified hericenone H.

Exemplary compositions of a monoalkyl tryptamine compounds of the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone in exemplary molar ratios are shown in Table 1. A monoalkyl tryptamine compound of the disclosure may be any one of the exemplary embodiments described above including their crystalline forms as disclosed herein.

TABLE 1

| | Molar ratio of a | Molar ratio of a | Molar ratio of a |
|---|---|---|---|
| | monoalkyl | monoalkyl | monoalkyl |
| | tryptamine | tryptamine | tryptamine |
| | compound:second | compound:second | compound:second |
| Second Compound | compound | compound | compound |
| 3,4- | About 1:100 to | About 1:25 to | About 1:5 to about |
| methylenedioxymethamphetamine | about 100:1 | about 25:1 | 5:1 |
| Citalopram | About 1:100 to | About 1:25 to | About 1:5 to about |
| | about 100:1 | about 25:1 | 5:1 |
| Escitalopram | About 1:100 to | About 1:25 to | About 1:5 to about |
| | about 100:1 | about 25:1 | 5:1 |
| Fluoxetine | About 1:100 to | About 1:25 to | About 1:5 to about |
| | about 100:1 | about 25:1 | 5:1 |
| Paroxetine | About 1:100 to | About 1:25 to | About 1:5 to about |
| | about 100:1 | about 25:1 | 5:1 |
| Sertraline | About 1:100 to | About 1:25 to | About 1:5 to about |
| | about 100:1 | about 25:1 | 5:1 |
| [3-(2-Dimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxytryptamine | About 1:100 to | About 1:25 to | About 1:5 to about |
| | about 100:1 | about 25:1 | 5:1 |
| 4-hydroxy-N,N-dimethyltryptamine | About 1:100 to | About 1:25 to | About 1:5 to about |
| | about 100:1 | about 25:1 | 5:1 |
| [3-(2-methylaminoethyl)-1H-indol- | About 1:100 to | About 1:25 to | About 1:5 to about |
| 4-yl] dihydrogen phosphate | about 100:1 | about 25:1 | 5:1 |
| 4-hydroxy-N-methyltryptamine | About 1:100 to | About 1:25 to | About 1:5 to about |
| | about 100:1 | about 25:1 | 5:1 |
| [3-(aminoethyl)-1H-indol-4-yl] | About 1:100 to | About 1:25 to | About 1:5 to about |
| dihydrogen phosphate | about 100:1 | about 25:1 | 5:1 |
| [3-(2-trimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxy-N,N,N- | About 1:100 to | About 1:25 to | About 1:5 to about |
| trimethyltryptamine | about 100:1 | about 25:1 | 5:1 |
| THC | About 1:100 to | About 1:25 to | About 1:5 to about |
| | about 100:1 | about 25:1 | 5:1 |
| CBC | About 1:100 to | About 1:25 to | About 1:5 to about |
| | about 100:1 | about 25:1 | 5:1 |
| CBD | About 1:100 to | About 1:25 to | About 1:5 to about |
| | about 100:1 | about 25:1 | 5:1 |
| CBG | About 1:100 to | About 1:25 to | About 1:5 to about |
| | about 100:1 | about 25:1 | 5:1 |
| Myrcene | About 1:100 to | About 1:25 to | About 1:5 to about |
| | about 100:1 | about 25:1 | 5:1 |
| Pinene | About 1:100 to | About 1:25 to | About 1:5 to about |
| | about 100:1 | about 25:1 | 5:1 |
| Caryophyllene | About 1:100 to | About 1:25 to | About 1:5 to about |
| | about 100:1 | about 25:1 | 5:1 |
| Limonene | About 1:100 to | About 1:25 to | About 1:5 to about |
| | about 100:1 | about 25:1 | 5:1 |
| Humulene | About 1:100 to | About 1:25 to | About 1:5 to about |
| | about 100:1 | about 25:1 | 5:1 |
| Linalool | About 1:100 to | About 1:25 to | About 1:5 to about |
| | about 100:1 | about 25:1 | 5:1 |
| Adrenaline | About 1:100 to | About 1:25 to | About 1:5 to about |
| | about 100:1 | about 25:1 | 5:1 |
| Amineptine | About 1:100 to | About 1:25 to | About 1:5 to about |
| | about 100:1 | about 25:1 | 5:1 |
| Erinacine A | About 1:100 to | About 1:25 to | About 1:5 to about |
| | about 100:1 | about 25:1 | 5:1 |
| Hericenone A | About 1:100 to | About 1:25 to | About 1:5 to about |
| | about 100:1 | about 25:1 | 5:1 |
| Phenelzine | About 1:100 to | About 1:25 to | About 1:5 to about |
| | about 100:1 | about 25:1 | 5:1 |

Exemplary pharmaceutical compositions of a monoalkyl tryptamine compound of the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and an excipient with exemplary molar ratios of a monoalkyl tryptamine compound to the second compound are shown in Table 2. A monoalkyl tryptamine compound of the disclosure may be any one of the exemplary embodiments described above including their crystalline forms as disclosed herein.

TABLE 2

| | Molar ratio of a | Molar ratio of a | Molar ratio of a |
|---|---|---|---|
| | monoalkyl | monoalkyl | monoalkyl |
| | tryptamine | tryptamine | tryptamine |
| | compound:second | compound:second | compound:second |
| Second Compound | compound | compound | compound |
| 3,4- | About 1:100 to | About 1:25 to | About 1:5 to about |
| methylenedioxymethamphetamine | about 100:1 | about 25:1 | 5:1 |
| Citalopram | About 1:100 to | About 1:25 to | About 1:5 to about |
| | about 100:1 | about 25:1 | 5:1 |
| Escitalopram | About 1:100 to | About 1:25 to | About 1:5 to about |
| | about 100:1 | about 25:1 | 5:1 |
| Fluoxetine | About 1:100 to | About 1:25 to | About 1:5 to about |
| | about 100:1 | about 25:1 | 5:1 |
| Paroxetine | About 1:100 to | About 1:25 to | About 1:5 to about |
| | about 100:1 | about 25:1 | 5:1 |
| Sertraline | About 1:100 to | About 1:25 to | About 1:5 to about |
| | about 100:1 | about 25:1 | 5:1 |
| [3-(2-Dimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxytryptamine | About 1:100 to | About 1:25 to | About 1:5 to about |
| | about 100:1 | about 25:1 | 5:1 |
| 4-hydroxy-N,N-dimethyltryptamine | About 1:100 to | About 1:25 to | About 1:5 to about |
| | about 100:1 | about 25:1 | 5:1 |
| [3-(2-methylaminoethyl)-1H-indol- | About 1:100 to | About 1:25 to | About 1:5 to about |
| 4-yl] dihydrogen phosphate | about 100:1 | about 25:1 | 5:1 |
| 4-hydroxy-N-methyltryptamine | About 1:100 to | About 1:25 to | About 1:5 to about |
| | about 100:1 | about 25:1 | 5:1 |
| [3-(aminoethyl)-1H-indol-4-yl] | About 1:100 to | About 1:25 to | About 1:5 to about |
| dihydrogen phosphate | about 100:1 | about 25:1 | 5:1 |
| [3-(2-trimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxy-N,N,N- | About 1:100 to | About 1:25 to | About 1:5 to about |
| trimethyltryptamine | about 100:1 | about 25:1 | 5:1 |
| THC | About 1:100 to | About 1:25 to | About 1:5 to about |
| | about 100:1 | about 25:1 | 5:1 |
| CBC | About 1:100 to | About 1:25 to | About 1:5 to about |
| | about 100:1 | about 25:1 | 5:1 |
| CBD | About 1:100 to | About 1:25 to | About 1:5 to about |
| | about 100:1 | about 25:1 | 5:1 |
| CBG | About 1:100 to | About 1:25 to | About 1:5 to about |
| | about 100:1 | about 25:1 | 5:1 |
| Myrcene | About 1:100 to | About 1:25 to | About 1:5 to about |
| | about 100:1 | about 25:1 | 5:1 |
| Pinene | About 1:100 to | About 1:25 to | About 1:5 to about |
| | about 100:1 | about 25:1 | 5:1 |
| Caryophyllene | About 1:100 to | About 1:25 to | About 1:5 to about |
| | about 100:1 | about 25:1 | 5:1 |
| Limonene | About 1:100 to | About 1:25 to | About 1:5 to about |
| | about 100:1 | about 25:1 | 5:1 |
| Humulene | About 1:100 to | About 1:25 to | About 1:5 to about |
| | about 100:1 | about 25:1 | 5:1 |
| Linalool | About 1:100 to | About 1:25 to | About 1:5 to about |
| | about 100:1 | about 25:1 | 5:1 |
| Adrenaline | About 1:100 to | About 1:25 to | About 1:5 to about |
| | about 100:1 | about 25:1 | 5:1 |
| Amineptine | About 1:100 to | About 1:25 to | About 1:5 to about |
| | about 100:1 | about 25:1 | 5:1 |
| Erinacine A | About 1:100 to | About 1:25 to | About 1:5 to about |
| | about 100:1 | about 25:1 | 5:1 |
| Hericenone A | About 1:100 to | About 1:25 to | About 1:5 to about |
| | about 100:1 | about 25:1 | 5:1 |
| Phenelzine | About 1:100 to | About 1:25 to | About 1:5 to about |
| | about 100:1 | about 25:1 | 5:1 |

An “effective amount” or a “therapeutically effective amount” of a monoalkyl tryptamine compound of the disclosure is generally in the range of about 0.1 to about 100 mg daily (oral dose), of about 0.1 to about 50 mg daily (oral dose) of about 0.25 to about 25 mg daily (oral dose), of about 0.1 to about 5 mg daily (oral dose) or of about 0.5 to about 2.5 mg daily (oral dose). The actual amount required for treatment of any particular patient may depend upon a variety of factors including, for example, the disease being treated and its severity; the specific pharmaceutical composition employed; the age, body weight, general health, sex, and diet of the patient; the mode of administration; the time of administration; the route of administration; and the rate of excretion; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173 (2001), which is incorporated herein by reference. A monoalkyl tryptamine compound of the disclosure and pharmaceutical compositions containing it may be used in combination with other agents that are generally administered to a patient being treated for psychological and other disorders discussed above. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. Exemplary carriers include those that do not substantially alter the structure or activity of monoalkyl tryptamine compound of the disclosure, nor produce undesirable biological effects or otherwise interact in a deleterious manner with any other component(s) of the pharmaceutical composition.

The pharmaceutical compositions of the disclosure may be prepared by methods know in the pharmaceutical formulation art, for example, see Remington's Pharmaceutical Sciences, 18th Ed., (Mack Publishing Company, Easton, Pa., 1990), which is incorporated herein by reference. In a solid dosage form, a 4-HO-DPT compound of the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alignates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like, (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

In some embodiments, the excipient is not water. In some embodiments, the excipient is not a solvent (e.g., EtOH, diethyl ether, ethyl acetate, or hydrocarbon-based solvents (e.g., hexanes). In some embodiments, the dosage form is substantially free of water and/or solvents, for example less than about 5% water by mass, less than 2% water by mass, less than 1% water by mass, less than 0.5% water by mass, or less than 0.1% water by mass.

Excipients or pharmaceutically acceptable adjuvants known in the pharmaceutical formulation art may also be used in the pharmaceutical compositions of the disclosure. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the disclosure may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others well known in the art. They may contain pacifying agents and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Solid dosage forms for oral administration, which includes capsules, tablets, pills, powders, and granules, may be used. In such solid dosage forms, the active compound may be mixed with at least one inert, pharmaceutically acceptable excipient (also known as a pharmaceutically acceptable carrier).

Administration of monoalkyl tryptamine compounds of the disclosure in pure form or in an appropriate pharmaceutical composition may be carried out via any of the accepted modes of administration or agents for serving similar utilities. Thus, administration may be, for example, orally, buccally, nasally, parenterally (intravenous, intramuscular, or subcutaneous), topically, transdermally, intravaginally, intravesically, or intrasystemically, in the form of solid, semi-solid, lyophilized powder, or liquid dosage forms, such as, for example, tablets, suppositories, pills, soft elastic and hard gelatin capsules, powders, solutions, suspensions, or aerosols, or the like, such as, for example, in unit dosage forms suitable for simple administration of precise dosages. One route of administration may be oral administration, using a convenient daily dosage regimen that can be adjusted according to the degree of severity of the disease-state to be treated.

### EXAMPLES

The preparation of compounds of formula (I) are discussed below.

### Example 1: Direct Alkylation Preparation of Compounds of Formula (I)

Mono-substituted amines of formula (I) may be prepared by treating the corresponding primary amine with an alkyl halide or an alkenyl halide (R₁Br, e.g., ethyl bromide) in a suitable solvent to provide a monoalkylated product (shown below). Subsequent treatment by a base yields the free base of formula (I). Exemplary compounds of formula (I) are listed in Table 3.

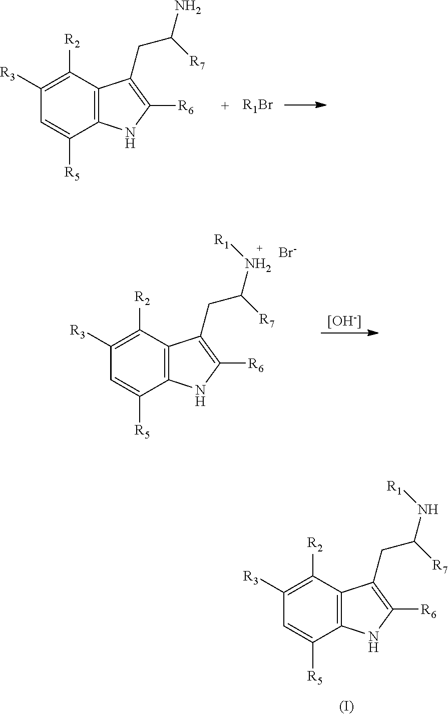

TABLE 3

| Compound | R₁ | R₂ | R₃ | R₅ | R₆ | R₇ |
|---|---|---|---|---|---|---|
| 1 | Ethyl | OH | H | H | H | H |
| 2 | Propyl | OH | H | H | H | H |
| 3 | isopropyl | OH | H | H | H | H |
| 4 | n-butyl | OH | H | H | H | H |
| 5 | Vinyl | OH | H | H | H | H |
| 6 | Allyl | OH | H | H | H | H |
| 7 | 2-butenyl | OH | H | H | H | H |
| 8 | Methyl | OCH₃ | H | H | H | H |
| 9 | Ethyl | OCH₃ | H | H | H | H |
| 10 | Propyl | OCH₃ | H | H | H | H |
| 11 | isopropyl | OCH₃ | H | H | H | H |
| 12 | n-butyl | OCH₃ | H | H | H | H |
| 13 | Vinyl | OCH₃ | H | H | H | H |
| 14 | Allyl | OCH₃ | H | H | H | H |
| 15 | 2-butenyl | OCH₃ | H | H | H | H |
| 16 | Methyl | —OC(O)CH₃ | H | H | H | H |
| 17 | Ethyl | —OC(O)CH₃ | H | H | H | H |
| 18 | Propyl | —OC(O)CH₃ | H | H | H | H |
| 19 | isopropyl | —OC(O)CH₃ | H | H | H | H |
| 20 | n-butyl | —OC(O)CH₃ | H | H | H | H |
| 21 | Vinyl | —OC(O)CH₃ | H | H | H | H |
| 22 | Allyl | —OC(O)CH₃ | H | H | H | H |
| 23 | 2-butenyl | —OC(O)CH₃ | H | H | H | H |
| 24 | Methyl | —OC(O)OCH₃ | H | H | H | H |
| 25 | Ethyl | —OC(O)OCH₃ | H | H | H | H |
| 26 | Propyl | —OC(O)OCH₃ | H | H | H | H |
| 27 | isopropyl | —OC(O)OCH₃ | H | H | H | H |
| 28 | n-butyl | —OC(O)OCH₃ | H | H | H | H |
| 29 | Vinyl | —OC(O)OCH₃ | H | H | H | H |
| 30 | Allyl | —OC(O)OCH₃ | H | H | H | H |
| 31 | 2-butenyl | —OC(O)OCH₃ | H | H | H | H |
| 32 | Methyl | —OSO₂CH₃ | H | H | H | H |
| 33 | Ethyl | —OSO₂CH₃ | H | H | H | H |
| 34 | Propyl | —OSO₂CH₃ | H | H | H | H |
| 35 | isopropyl | —OSO₂CH₃ | H | H | H | H |
| 36 | n-butyl | —OSO₂CH₃ | H | H | H | H |
| 37 | Vinyl | —OSO₂CH₃ | H | H | H | H |
| 38 | Allyl | —OSO₂CH₃ | H | H | H | H |
| 39 | 2-butenyl | —OSO₂CH₃ | H | H | H | H |
| 40 | Methyl | H | OH | H | H | H |
| 41 | Ethyl | H | OH | H | H | H |
| 42 | Propyl | H | OH | H | H | H |
| 43 | isopropyl | H | OH | H | H | H |
| 44 | n-butyl | H | OH | H | H | H |
| 45 | Vinyl | H | OH | H | H | H |
| 46 | Allyl | H | OH | H | H | H |
| 47 | 2-butenyl | H | OH | H | H | H |
| 48 | Methyl | H | OCH₃ | H | H | H |
| 49 | Ethyl | H | OCH₃ | H | H | H |
| 50 | Propyl | H | OCH₃ | H | H | H |
| 51 | isopropyl | H | OCH₃ | H | H | H |
| 52 | n-butyl | H | OCH₃ | H | H | H |
| 53 | Vinyl | H | OCH₃ | H | H | H |
| 54 | Allyl | H | OCH₃ | H | H | H |
| 55 | 2-butenyl | H | OCH₃ | H | H | H |
| 56 | Methyl | H | —OC(O)CH₃ | H | H | H |
| 57 | Ethyl | H | —OC(O)CH₃ | H | H | H |
| 58 | Propyl | H | —OC(O)CH₃ | H | H | H |
| 59 | isopropyl | H | —OC(O)CH₃ | H | H | H |
| 60 | n-butyl | H | —OC(O)CH₃ | H | H | H |
| 61 | Vinyl | H | —OC(O)CH₃ | H | H | H |
| 62 | Allyl | H | —OC(O)CH₃ | H | H | H |
| 63 | 2-butenyl | H | —OC(O)CH₃ | H | H | H |
| 64 | Methyl | H | —OC(O)OCH₃ | H | H | H |
| 65 | Ethyl | H | —OC(O)OCH₃ | H | H | H |
| 66 | Propyl | H | —OC(O)OCH₃ | H | H | H |
| 67 | isopropyl | H | —OC(O)OCH₃ | H | H | H |
| 68 | n-butyl | H | —OC(O)OCH₃ | H | H | H |
| 69 | Vinyl | H | —OC(O)OCH₃ | H | H | H |
| 70 | Allyl | H | —OC(O)OCH₃ | H | H | H |
| 71 | 2-butenyl | H | —OC(O)OCH₃ | H | H | H |
| 72 | Methyl | H | —OSO₂CH₃ | H | H | H |
| 73 | Ethyl | H | —OSO₂CH₃ | H | H | H |
| 74 | Propyl | H | —OSO₂CH₃ | H | H | H |
| 75 | isopropyl | H | —OSO₂CH₃ | H | H | H |
| 76 | n-butyl | H | —OSO₂CH₃ | H | H | H |
| 77 | Vinyl | H | —OSO₂CH₃ | H | H | H |
| 78 | Allyl | H | —OSO₂CH₃ | H | H | H |
| 79 | 2-butenyl | H | —OSO₂CH₃ | H | H | H |

### Example 2: Reductive Amination Preparation of Compounds of Formula (I)

Monoalkylation may be accomplished via reductive amination (shown below) of the corresponding amine (R₁═H) with an appropriate aldehyde. Table 4 shows the aldehyde and corresponding R₁ group of compounds of formula (I).

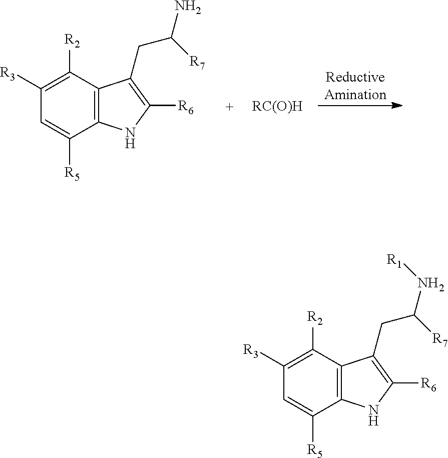

TABLE 4

| Aldehyde, RC(O)H | R₁ |
|---|---|
| HC(O)H | Methyl |
| CH₃C(O)H | Ethyl |
| CH₃CH₂C(O)H | Propyl |
| CH₃CH₂CH₂C(O)H | n-butyl |

### Example 3: Preparation of crystalline structure of 4-AcO-NMT chloride

Synthesis and Crystallization

The general procedure for the synthesis of 4-AcO-NMT chloride is shown by the reaction below.

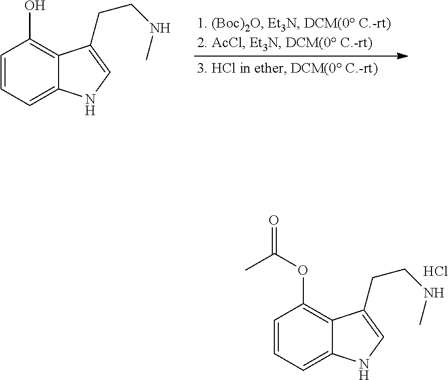 [SMILES: CNCCc1c[nH]c2cccc(O)c12 ; CNCCc1c[nH]c2cccc(OC(C)=O)c12 ; Cl]

4-AcO-NMT chloride was synthesized when 3-(2-(methylamino)ethyl)-1H-indol-4-ol (0.125 g, 0.65 mmol, 1 equiv) was added to a dry reaction vial containing anhydrous dichloromethane (DCM) (8 mL) under nitrogen and the contents were cooled to 0° C. under an ice-bath. Triethyl amine (1.2 equiv) was added to the resulting solution at 0° C. followed by Boc- anhydride (1.1 equiv) in a dropwise manner and the contents were then stirred at room temperature until the disappearance of the starting material (per TLC). After the reaction was completed the contents were diluted with 15 mL DCM and washed twice with cold water followed by brine. The organic layer was then dried under sodium sulfate and reduced under pressure to yield a solid (N-Boc-Norpsilocin) which was used in the next reaction without further purification.

Anhydrous DCM (10 mL) was added under nitrogen to a dry reaction vial containing the above obtained residue (N-Boc-Norpsilocin) and the contents were cooled to 0° C. under an ice-bath. Triethyl amine (2 equiv) was added to the resulting solution at 0° C. followed by acetyl chloride (1.5 equiv) in a dropwise manner and the contents were then stirred at room temperature until the disappearance of the starting material (per TLC). After the reaction completed the contents were diluted with 20 mL DCM and washed thrice with cold water followed by brine. The organic layer was then dried under sodium sulfate and reduced under pressure to yield semi solid residue (N-Boc-4-OAc-Norpsilocin). Thus, the obtained residue was diluted with anhydrous DCM (12 mL) and transferred to a reaction vial. HCl in ether (2N, 10 equiv) was added in a dropwise manner to the resulting solution at 0° C. The contents were then stirred at room temperature overnight until the disappearance of the starting material (per TLC). The resulting precipitate after the completion of the reaction was filtered and washed with cold DCM, diethyl ether and dried under vacuum to yield the desired compound as light-yellow solid with 44% overall yield.

¹H NMR (400 MHz, Deuterium Oxide) δ 7.37 (d, J=8.2 Hz, 1H), 7.22 (s, 1H), 7.17 (t, J=8.0 Hz, 1H), 6.80 (d, J=7.7 Hz, 1H), 3.31 (t, J=6.7 Hz, 2H), 3.03 (t, J=6.7 Hz, 2H), 2.61 (s, 3H), 2.37 (s, 3H).

The crystal for 4-AcO-NMT chloride was grown by the vapor diffusion of hexanes into a methylene chloride/methanol solution. Single crystal data, data collection and structure refinement details are summarized in Table 5.

TABLE 5

| | 4-AcO-NMT chloride |
|---|---|
| Chemical formula | Cl•C₁₃H₁₇N₂O₂ |
| Mᵣ | 268.73 |
| Crystal system, space group | Monoclinic, P2₁/c |
| Temperature (K) | 297 |
| a, b, c (Å) | 16.772 (4), 9.410 (3), 9.356(3) |
| β (°) | 105.241 (8) |
| V (Å³) | 1424.8 (7) |
| Z | 4 |
| Radiation type | Mo Kα |
| μ (mm⁻¹) | 0.27 |
| Crystal size (mm) | 0.4 × 0.39 × 0.09 |
| F(000) | 568 |
| Dₓ (Mg m⁻³) | 1.253 |
| λ (Å) | 0.71073 |
| θ (°) | 3.1-25.4 |
| PLATE | Colourless |
| Diffractometer | Bruker APEX-II CCD |
| Absorption correction | Absorption correction: multi-scan |
| | SADABS2016/2 (Bruker, 2016/2) |
| | was used for absorption |
| | correction. wR2(int) was 0.0848 |
| | before and 0.0650 after |
| | correction. The Ratio of minimum |
| | to maximum transmission is |
| | 0.8588. The λ/2 correction factor |
| | is Not present. |
| Tₘᵢₙ, Tₘₐₓ | 0.640, 0.745 |
| No. of measured, independent | 22348, 2705, 1907 |
| and observed | |
| [I > 2s(I)] reflections | |
| Rᵢₙₜ | 0.061 |
| θₘₐₓ, θₘᵢₙ (°) | 25.8, 3.1 |
| h | −20→20 |
| k | −11→11 |
| I | −11→11 |
| Refinement | F² |
| Least-squares matrix | Full |
| R[F² > 2σ(F²)], wR(F²), S | 0.044, 0.122, 1.09 |
| No. of reflections | 2705 |
| No. of parameters | 216 |
| No. of restraints | 72 |
| Hydrogen site location | Mixed |
| H-atom treatment | H atoms treated by a mixture of |
| | independent and constrained |
| | refinement |
| w | 1/[s²(Fₒ²) + (0.0413P)² + 0.877P] |
| | where P = (Fₒ² + 2F_{c}²)/3 |
| (Δ/σ)max | <0.001 |
| Δ [symbol] ₘₐₓ, Δ [symbol] ₘᵢₙ (e Å⁻³) | 0.22, −0.19 |
| Extinction Correction | SHELXL2018/3 (Sheldrick 2018), |
| | Fc* = kFc[1 + 0.001 × Fc²λ³/sin(2θ)]^{-1/4} |
| Extinction Coefficient | 0.0079 (16) |

Data collection: Bruker APEX3; cell refinement: Bruker SAINT; data reduction: Bruker SAINT; program(s) used to solve structure: SHELXS97 (Sheldrick 2008); program(s) used to refine structure: SHELXL 2018/3 (Sheldrick, 2015); molecular graphics: Olex2 1.3 (Dolomanov et al., 2009); software used to prepare material for publication: Olex2 1.3 (Dolomanov et al., 2009).

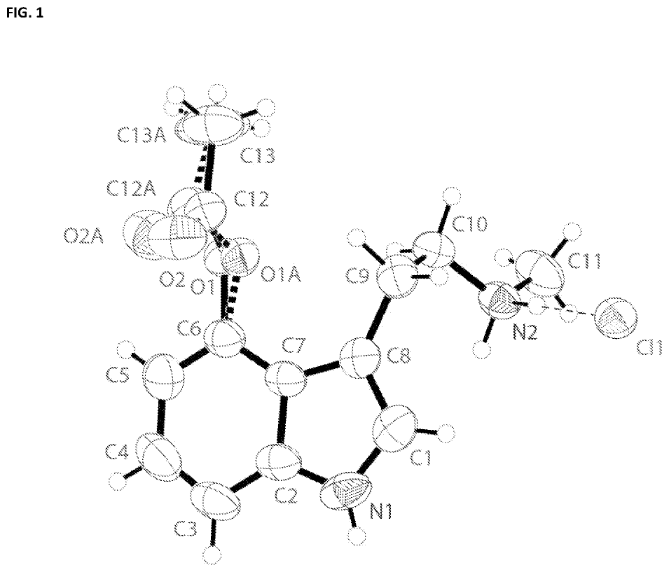

The crystalline structure of 4-AcO-NMT chloride is shown in FIG. 1.

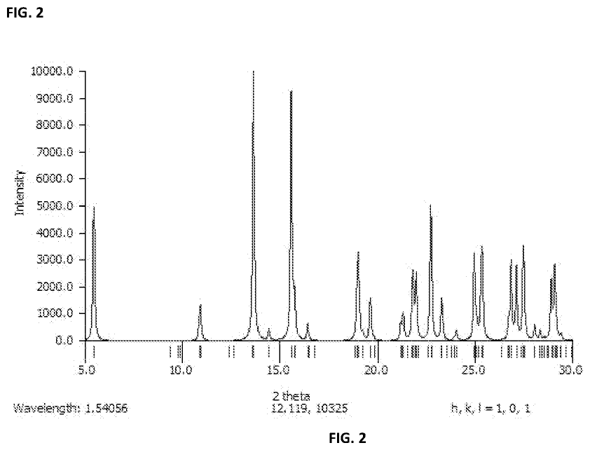

FIG. 2 is a simulated x-ray powder diffraction (XRPD) of crystalline 4-AcO-NMT chloride from its single crystal data. Crystalline 4-AcO-NMT chloride may be characterized by the XRPD peaks at 5.5, 13.6, and 15.6° 2θ±0.2° 2θ as well as by an XRPD pattern substantially similar to FIG. 2.

### REFERENCES

Bradley, R. J. & Johnston, V. S. (1970). Origin and Mechanism of Hallucinations, edited by W. Keup, pp. 333-344. New York: Plenum Press.
Cameron, L. P. & Olson, D. E. (2018). ACS Chem. Neurosci. 9, 2344-2357.
Carhart-Harris, R. L. & Goodwin, G. M. (2017). Neuropsychopharmacology, 42, 2105-2113.
Dinis-Oliveira, R. J. (2017). Drug Metab. Rev. 49, 84-91.
Johnson, M. W. & Griffiths, R. R. (2017). Neurotherapeutics 14, 734-740.
McKenna, D. J., Repke, D. B., Lo, L. & Peroutka, S. J. (1990). Neuropharmacology, 29, 193-198.
Nichols, D. E. (2012). WIREs Membr. Transp. Signal. 1, 559-579.
Repke, D. B., Grotjahn, D. B. & Shulgin, A. T. (1985). J. Med. Chem. 28, 892-896.
C. Lenz, J. Wick and D. Hoffmeister, J. Nat. Prod., 2017, 80, 2835-2838.
A. M. Sherwood, A. L. Halberstadt, A. K. Klein, J. D. McCorvy, K. W. Kaylo, R. B. Kargbo and P. Meisenheimer, J. Nat. Prod., Article ASAP, DOI: 10.1021/acs.jnatprod.9b01061.
N. Jensen, J. Gartz and H. Laatsch, Planta Med., 2006, 72, 665-666.
J. Gartz, Int. J. Crude Drug Res., 1989, 27, 141-144.
B. L. Roth, W. K. Kroeze, S. Patel and E. Lopez, The Neuroscientist, 2000, 6, 252-262.

1-138. (canceled)

139. A tryptamine compound selected from the group consisting of a compound of formula (I): 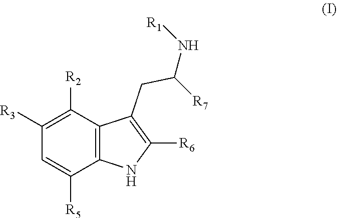
wherein:
R₁ is a straight chain or branched C₂-C₆ alkyl or a straight chain or branched C₂-C₆ alkenyl;
R₂ and R₃ are independently chosen from hydrogen, hydroxyl, —OR₉, —OC(O)R₈, or —OC(O)OR₄, —OSO₂R₄;
R₄ is a straight chain or branched C₁-C₆ alkyl or a substituted or unsubstituted aryl;
R₈ is a straight chain or branched C₁-C₆ alkyl or a substituted or unsubstituted aryl;
R₉ is a straight chain or branched C₁-C₆ alkyl or a substituted or unsubstituted aryl; and
R₅, R₆ and R₇ are each independently hydrogen or a straight chain or branched C₁-C₆ alkyl, or a pharmaceutically acceptable acid-addition salt thereof; and
a compound of formula (Ia): 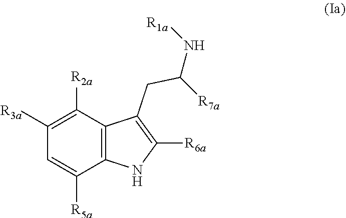
wherein:
R₁ₐ is a straight chain or branched C₁-C₆ alkyl or a straight chain or branched C₂-C₆ alkenyl;
one of R₂ₐ and R₃ₐ is hydrogen and the other of R₂ₐ and R₃ₐ is selected from —OC(O)R₈ₐ, —OC(O)OR₄ₐ, and —OSO₂R₄ₐ;
R₄ₐ is a straight chain or branched C₁-C₆ alkyl or a substituted or unsubstituted aryl;
R₈ₐ is selected from straight chain or branched C₁-C₆ alkyl;
R₅ₐ, R₆ₐ and R₇ₐ are each independently hydrogen or a straight chain or branched C₁-C₆ alkyl, or a pharmaceutically acceptable acid-addition salt thereof.

140. The tryptamine compound of claim 139, wherein the tryptamine compound is a compound of formula (I) and R₁ is selected from the group consisting of butyl, 2-butenyl, ethyl, isopropyl, propyl, allyl, vinyl, 1-methylethylidenyl, and 3-pentanyl.

141. The tryptamine compound of claim 139, wherein the tryptamine compound is a compound of formula (Ia) and R₁ₐ is selected from the group consisting of methyl, butyl, 2-butenyl, ethyl, isopropyl, propyl, allyl, vinyl, 1-methylethylidenyl, and 3-pentanyl.

142. The tryptamine compound of claim 139, wherein the tryptamine compound is a compound of formula (I) and one of R₂ and R₃ is hydrogen and the other of R₂ and R₃ is selected from the group consisting of pivaloyloxyl, acetoxyl, methylcarbonato, hydroxyl, (methylsulfonyl)oxyl, (benzyl)oxyl, and methoxyl.

143. The tryptamine compound of claim 139, wherein the tryptamine compound is a compound of formula (Ia) and one of R₂ₐ and R₃ₐ is hydrogen and the other of R₂ₐ and R₃ₐ is selected from the group consisting of pivaloyloxyl, acetoxyl, methylcarbonato, hydroxyl, (methylsulfonyl)oxyl, (benzyl)oxyl, and methoxyl.

144. A tryptamine compound selected from the group consisting of a compound of formula (II): 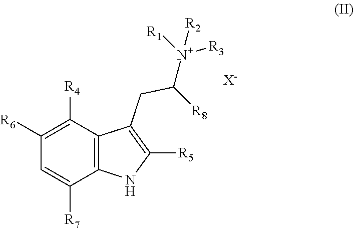
wherein:
R₁ is a straight chain or branched C₁-C₆ alkyl or a straight chain or branched C₂-C₆ alkenyl;
R₂ and R₃ are both hydrogen;
R₄ and R₆ are independently chosen from hydrogen, hydroxyl, —OR₅, —OC(O)R₁₁, —OC(O)OR₁₂, —OSO₂R₁₂;
R₅ is a straight chain or branched C₁-C₆ alkyl or a substituted or unsubstituted aryl;
R₁₁ is a straight chain or branched C₁-C₆ alkyl or a substituted or unsubstituted aryl;
R₁₂ is a straight chain or branched C₁-C₆ alkyl or a substituted or unsubstituted aryl;
R₇, R₈ and R₉ are each independently hydrogen or a straight chain or branched C₁-C₆ alkyl; and
X⁻ is a pharmaceutically acceptable anion; and
a compound of formula (IIa): 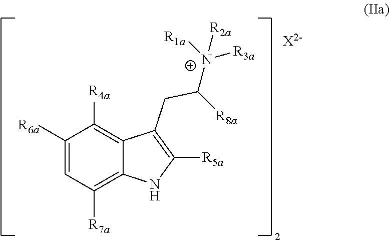
wherein:
R^{1a} is a straight chain or branched C₁-C₆ alkyl or a straight chain or branched C₂-C₆ alkenyl;
R₂ₐ and R₃ₐ are both hydrogen;
R₄ₐ and R₆ₐ are independently chosen from hydrogen, hydroxyl, —OR₅ₐ, —OC(O)R₁₁ₐ, —OC(O)OR₁₂a, —OSO₂R₁₂ₐ;
R₅ₐ is a straight chain or branched C₁-C₆ alkyl or a substituted or unsubstituted aryl;
R₁₁ₐ is a straight chain or branched C₁-C₆ alkyl or a substituted or unsubstituted aryl;
R₁₂ₐ is a straight chain or branched C₁-C₆ alkyl or a substituted or unsubstituted aryl;
R₇ₐ, R₈ₐ and R₉ₐ are each independently hydrogen or a straight chain or branched C₁-C₆ alkyl; and
X²⁻ is a pharmaceutically acceptable dianion.

145. The tryptamine compound of claim 144, wherein the tryptamine compound is a compound of formula (II) and R₁ is selected from the group consisting of methyl, butyl, 2-butenyl, ethyl, isopropyl, propyl, allyl, vinyl, 1-methylethylidenyl, and 3-pentanyl.

146. The tryptamine compound of claim 144, wherein the tryptamine compound is a compound of formula (IIa) and R₁ₐ is selected from the group consisting of methyl, butyl, 2-butenyl, ethyl, isopropyl, propyl, allyl, vinyl, 1-methylethylidenyl, and 3-pentanyl.

147. The tryptamine compound of claim 144, wherein the tryptamine compound is a compound of formula (II) and one of R₄ and R₆ is hydrogen and the other of R₄ and R₆ is selected from the group consisting of pivaloyloxyl, acetoxyl, methylcarbonato, hydroxyl, (methylsulfonyl)oxyl, (benzyl)oxyl, and methoxyl.

148. The tryptamine compound of claim 144, wherein the tryptamine compound is a compound of formula (IIa) and one of R₄ₐ and R₆ₐ is hydrogen and the other of R₄ₐ and R₆ₐ is selected from the group consisting of pivaloyloxyl, acetoxyl, methylcarbonato, hydroxyl, (methylsulfonyl)oxyl, (benzyl)oxyl, and methoxyl.

149. A composition comprising a therapeutically effective amount of a tryptamine compound of claim 139 and a pharmaceutically acceptable excipient.

150. A composition comprising a therapeutically effective amount of a tryptamine compound of claim 144 and a pharmaceutically acceptable excipient.

151. A composition comprising a first active component: a tryptamine compound of claim 139; and a second active component selected from the group consisting of (a) a serotonergic drug, (b) a purified psilocybin derivative, (c) a purified cannabinoid, (d) a monoamine oxidase inhibitor, (e) a purified terpene, (f) a purified erinacine, (g) a purified hericenone, and (h) a purified monoamine oxidase inhibitor.

152. A composition comprising a first active component: a tryptamine compound of claim 144; and a second active component selected from the group consisting of (a) a serotonergic drug, (b) a purified psilocybin derivative, (c) a purified cannabinoid, (d) a monoamine oxidase inhibitor, (e) a purified terpene, (f) a purified erinacine, (g) a purified hericenone, and (h) a purified monoamine oxidase inhibitor.

153. A method of preventing or treating a psychological disorder comprising:
identifying a subject in need of treatment or prevention; and
administering to a subject in need thereof a therapeutically effective amount of the compound of claim 139.

154. 2-[4-(acetyloxy)-1H-indol-3-yl]ethyl}(methyl)azanium chloride (4-AcO-NMT chloride).

155. Crystalline 2-[4-(acetyloxy)-1H-indol-3-yl]ethyl}(methyl)azanium chloride (4-AcO-NMT chloride).

156. Crystalline 4-AcO-NMT chloride according to claim 155, characterized by at least one of:
a monoclinic, P2₁/c crystal system space group at a temperature of about 297 K;
a unit cell dimensions a=16.772 (4) Å, b=9.410 (3) Å, c=9.356 (3) Å, and β=105.241 (8°);
an XRPD having peaks at 5.5, 13.6, and 15.6° 2θ±0.2° 200; or
an XRPD pattern substantially similar to FIG. 2.

157. A composition comprising a therapeutically effective amount of crystalline 4-AcO-NMT chloride of claim 155 and a pharmaceutically acceptable excipient.

158. A composition comprising a first active component: crystalline 4-AcO-NMT chloride of claim 155; and a second active component selected from the group consisting of (a) a serotonergic drug, (b) a purified psilocybin derivative, (c) a purified cannabinoid, (d) a monoamine oxidase inhibitor, (e) a purified terpene, (f) a purified erinacine, (g) a purified hericenone, and (h) a purified monoamine oxidase inhibitor.
